(19) United States
(12) Patent Application Publication
(10) Pub. No.: US 2015/0241445 A1
(43) Pub. Date: Aug. 27, 2015

(54) COMPOSITIONS AND METHODS OF PROGNOSIS AND CLASSIFICATION FOR RECOVERY FROM SURGICAL TRAUMA

(71) Applicant: The Board of Trustees of the Leland Stanford Junior University, Palo Alto, CA (US)

(72) Inventors: Brice L. Gaudilliere, Palo Alto, CA (US); Gabriela K. Fragiadakis, Stanford, CA (US); Martin S. Angst, Stanford, CA (US); Garry P. Nolan, Redwood City, CA (US)

(21) Appl. No.: 14/629,262

(22) Filed: Feb. 23, 2015

Related U.S. Application Data
(60) Provisional application No. 61/943,941, filed on Feb. 24, 2014.
(60) Provisional application No. 62/007,330, filed on Jun. 3, 2014.

(51) Int. Cl. G01N 33/68

(52) U.S. Cl. CPC G01N 33/6863; G01N 2800/52; G01N 2570/00

(57) ABSTRACT

Multiparametric analysis at the single cell level of biological samples obtained from an individual undergoing surgery is used to obtain a determination of changes in immune cell subsets, which changes include, without limitation, altered activation states of proteins involved in signaling pathways. Changes occur in signaling pathways of these immune cells that are predictive of the recovery status of the individual.

### GOVERNMENT SUPPORT

This invention was made with government support under Grant No. HV000242 awarded by the National Institutes of Health. The Government has certain rights in the invention.

### BACKGROUND OF THE INVENTION

More than 40 million surgeries are performed annually in the US alone. This number is expected to grow as the proportion of elderly patients is increasing. Convalescence after surgery is highly variable, and delayed recovery causes substantial personal suffering as well as major societal and economic costs. Recent efforts in perioperative care have partially addressed this challenge by implementing enhanced-recovery protocols, evidenced-based practice guidelines that are largely anchored in observational data. A better understanding of the mechanisms driving recovery after surgery will advance current strategies, and allow tailoring them to patient-specific and procedural needs.

Tissue injury produces a profound inflammatory response, which explains the long-standing interest in identifying immune mechanisms determining recovery from surgical trauma. Previous studies predominantly focused on secreted humoral factors, distribution patterns of immune cell subsets or genomic analysis of pooled circulating leukocytes. While these studies provided important insight into mechanisms governing the inflammatory response to surgery, they did not report strong immune correlates of clinical recovery.

Importantly, available platforms did not allow examination of functional in-vivo responses of immune cell subsets directly at the single cell level, which may explain these findings. Thus, there is a need for improved measures for the diagnosis, prognosis, treatment, management, and therapeutic development for recovery from surgical trauma.

### SUMMARY OF THE INVENTION

Compositions and methods are provided for classification, diagnosis, prognosis, theranosis, and/or prediction of an outcome following surgery in a subject. In some embodiments, the methods comprise the steps of obtaining a biological sample from a patient contemplating or undergoing surgery, measuring single cell levels of activated signaling proteins in immune cell subsets involved in response to surgical trauma, e.g. inflammatory response, determining whether changes in signaling responses associated with recovery are present, and providing an assessment of the patient's prognosis for time to recovery. The sample may be activated ex vivo, or activated in vivo, e.g. during surgery.

In some embodiments, the intracellular signaling pathways involve changes in the phosphorylation of proteins involved in intracellular signaling pathways. Changes in the distribution of immune cell subsets can also be monitored. The predictive changes in signaling molecules can be observed within about 72 hours after surgery, within about 48 hours after surgery, within about 24 hours after surgery, and may be observed within about 1 hour after surgery; or alternatively can be observed after ex vivo activation of a cell sample obtained before or after surgery. While later occurring changes may be of interest, in general analysis shortly after surgery or ex vivo activation provides benefits for appropriate adjustments to patient care.

In some embodiments, changes are measured in single cells of phosphorylated protein components of intracellular signaling pathways, which proteins are present in specific immune cell subsets. Changes after ex vivo activation or within about 24 hours following surgery, e.g. when compared to a baseline pre-surgery level, or to a baseline level shortly following surgery, can be predictive of the time to recovery for the individual. This information can be provided to the individual or care-giver. In particular, analysis of cells based on ex vivo activation can be used to inform about the risk of surgery for the subject and to make decisions regarding whether to undergo surgery.

In particular, it is shown that the time to recovery, for example as measured by time to 50% global functioning; time to mild pain; time to mild functional impairment, etc. (which may be referred to as time to recovery parameters), is correlated with changes in phosphorylation of intracellular signaling pathway proteins present in circulating monocytes, e.g. in CD14⁺ monocytes. Signaling responses of interest include a significant change from baseline in, for example, a protein of the pNF-κB (pP65) signaling pathway, a protein of pCREB signaling pathway, a protein on pSTAT3 signaling pathway. In some embodiments, measurement is made at a single cell level of one or more of pNF-κB (pP65), pCREB and pSTAT3 at a baseline time point prior to or shortly surgery. Changes in signal intensity of these proteins in monocyte populations is correlated with the patient's time to recovery, allowing a distinction between individuals who have a high probability of rapid recovery from those who have a low probability of rapid recovery. Assessment in a patient allows improved care and decision-making, where patients classified according to probability of recovery time can be treated appropriately, e.g. more supportive care, longer time in a managed care facility, delay of elective surgery, and the like. Appropriate care can reduce, for example readmission for individuals following surgery.

In some embodiments, the monocyte population that is monitored for changes in signaling pathways is a CD14 positive population. Further classification, or gating of the cell populations for analysis, can utilize markers comprising one or more of CD66, CD3, CD11b, and HLA-DR. A monocyte population of interest is CD66 negative (CD66⁻); CD3 negative (CD3⁻); CD11b positive (CD11b⁺); and HLA-DR positive, although the expression of HLA-DR can be low or moderate.

In one embodiment of the invention, the methods of determining time to recovery status in a patient following surgery comprises obtaining a patient sample(s) comprising circulating immune cells prior to surgery. Blood samples are a convenient source of circulating immune cells, particularly whole blood, although PBMC fractions also find use. The patient sample is stimulated ex vivo with an effective dose of an agent that stimulates CD14⁺ monocytes, including without limitation agents that stimulate toll-like receptors (TLRs). In other embodiments, one or more patient sample(s) comprising circulating immune cells, usually a time course of samples from a baseline to a time point within about 1 hour to about 72 hours following surgery.

The sample(s) is physically contacted with a panel of affinity reagents specific for signaling proteins and for markers that distinguish subsets of immune cells. Usually the affinity reagents comprise a detectable label, e.g. isotope, fluorophore, etc. Signal intensity of the markers is measured, preferably at a single cell level. Suitable methods of analysis include, without limitation, flow cytometry, mass cytometry, confocal microscopy, and the like. The data, which can include measurements of monocyte cell population size, intensity of signaling molecules in selected immune cell subsets, etc., is compared to measurements of the same from the baseline cell population. The data can be normalized for comparison.

Quantitation of one or more of pNF-κB (pP65), pCREB and pSTAT3 in CD14⁺ monocytes is of particular interest, where the sample may be pre-surgery in the absence or presence of ex vivo activation; and/or 1 hour post-surgery, 2 hours post surger, 4 hours post-surgery, and within about 24, about 48, about 72 hours post-surgery. pCREB AND pNFkB decrease on average from baseline to 1 h. A lower pCREB signal at 1 h compared to baseline indicates a more rapid recovery from functional impairment of the hip. A lower pNF-κB signal at 1 h compared to baseline indicates a more rapid recovery from pain. The greater the decrease in STAT3 between 1 h and 24 h the faster patients return to 50% of global functioning. In some embodiments, two or more of pNF-κB (pP65), pCREB and pSTAT3 in CD14⁺ monocytes are monitored. In cells activated ex vivo, phosphorylation of MAPKAPK2 is of particular interest, where individuals with a lower increase of pMAPKAPK2 relative to a control indicates a more rapid recovery from functional impairment.

In other embodiments of the invention a device or kit is provided for the analysis of patient samples. Such devices or kits will include reagents that specifically identify one or more cells and signaling proteins indicative of the time to recovery status of the patient, including without limitation affinity reagents specific for one or more of pNF-κB (pP65), pCREB, pSTAT3. Affinity reagents may further comprise a reagent specific for CD14; and can further comprise reagents specific for one or more of CD66, CD3, CD11b, and HLA-DR. In some embodiments the affinity reagents comprise one or more additional specificities from the panels set forth in Table 2. In some embodiments the affinity reagents are antibodies. In some embodiments the affinity reagents comprise a detectable label. The reagents can be provided as a kit comprising reagents in a suspension or suspendable form, e.g. reagents suitable for flow or mass cytometry, and the like. A kit may also include an activator suitable for use ex vivo, including without limitations a TLR4 agonist, e.g. lipopolysaccharides (LPS); paclitaxel; heat shock proteins, (HSP22, 60, 70, 72, Gp96); high mobility group proteins (HMGB1); proteoglycans (versican, heparin sulfate, hyaluronic acid fragments); fibronectin, tenascin-C; etc.

The reagents can be provided in isolated form, or pre-mixed as a cocktail suitable for the methods of the invention. A kit can include instructions for using the plurality of reagents to determine data from the sample; and instructions for statistically analyzing the data. The kits may be provided in combination with a system for analysis, e.g. a system implemented on a computer. Such a system may include a software component configured for analysis of data obtained by the methods of the invention.

Also described herein is a method for assessing prognosis for time to recovery of a patient following surgery, comprising: obtaining a dataset associated with a sample obtained from the subject, wherein the dataset comprises quantitative data for the signaling response of specific immune cell subsets comprising data for at least one of one or more of pNF-κB (pP65), pCREB and pSTAT3; and analyzing the dataset for changes at the single cell level for these markers, wherein a statistically significant match with an extended recovery pattern is indicative of the time to recovery of the subject. The data may be analyzed by a computer processor. The processor may be communicatively coupled to a storage memory for analyzing the data. Also described herein is a computer-readable storage medium storing computer-executable program code, the program code comprising: program code for storing and analyzing data obtained by the methods of the invention.

In an embodiment, the method further comprises selecting a treatment regimen for the surgical patient based on the analysis. In an embodiment, the method further comprises determining a treatment course for the subject based on the analysis.

Treatment regimens of interest can include decision-making for proceeding with elective surgery, extended hospital stay, extended care at an intermediate facility, increased post-surgery follow-up, and the like. Treatment regimens of interest may also include administration of a therapeutic agent that decreases the activation of CD14⁺ monocytes.

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The patent or application file contains at least one drawing executed in color. Copies of this patent or application publication with color drawings will be provided by the Office upon request and payment of the necessary fee.

FIG. 1: Mass tag barcoding enables the longitudinal analysis of the cellular immune response in peripheral blood of patients undergoing surgery. (a) Experimental workflow. Whole blood samples from six patients undergoing primary hip arthroplasty were collected 1 h before surgery (baseline, BL), and 1 h, 24 h, 72 h, and 6 weeks after surgery. Following red blood cell lysis, leukocyte samples from each patient were barcoded using a unique combination of palladium isotopes (panel 1). Barcoded samples were pooled, stained with a panel of 31 antibodies (panel 2, Table 2), and analyzed by mass cytometry (panel 3). Raw mass cytometry data were normalized for signal variation over time₅₄ (panel 4), de-barcoded (panel 5) and analyzed (panel 6). (b) Assay validation in surgical patients. Ten intracellular signaling responses to surgery were quantified for four immune cell subsets (neutrophils, CD14+ MCs, CD4+ and CD8+ T cells). Signal induction for each signaling molecule was calculated as the difference of inverse hyperbolic sine medians between samples obtained at baseline and at 1 h, 24 h, 72 h, and 6 weeks after surgery (“arcsinh ratio”). Five of 10 phospho-proteins (pSTAT1, pSTAT3, pSTAT5, pCREB, pP38) displayed reproducible changes at 1 h, 24 h, or 72 h after surgery compared to baseline. Results are shown as means±SEM. SAM Two class paired was used for statistical analysis (** indicates a false discovery rate q<0.01).

FIG. 2: Surgery induces a redistribution of major immune cell-types and a 6-fold expansion of HLA-DR^{low} CD14+ monocytes. (a) Frequencies of neutrophils, CD14+MCs, cDCs, pDCs, NK cells, B cells, CD4+ T cells, and CD8+ T cells are depicted for 26 patients 1 h, 24 h, 72 h, and 6 weeks after surgery. Cell-types were identified by manual gating (FIG. 7). Neutrophil frequency was quantified as percent of total hematopoietic cells (CD61⁻CD235⁻). All other cell frequencies are expressed as percent total of mononuclear cells (CD45+CD66−). Significant changes occurred for all cell types (**q<0.01, SAM Two class paired). Results are shown as mean fold change (±SEM). (b) Visual representation of unsupervised hierarchical clustering. Results are shown for CD45+CD66− immune cells. The analysis used 21 cell surface markers (Table 2). Major immune cell compartments are contoured (FIG. 10). Contoured in red are CD14+MCs. The color scale indicates median intensity of CD14 expression. (c) CD14+MCs were clustered into HLA-DR^{hi} (yellow), HLA-DR^{mid} (green), and HLA-DR^{low} (blue) subsets. The color scale indicates the median intensity of HLA-DR expression. (d-g). Histogram plots. Arrows designate histograms of HLA-DR expression for CD14⁺MC clusters (red) against HLA-DR background expression in all CD45+CD66− cells (blue). (h-k). CD14⁺MC cell cluster frequencies 1 h before and 1 h, 24 h, and 72 h after surgery. Expansion of all CD14⁺MC clusters (h) was attributable to the expansion of the HLA-DRmid (j) and HLA-DRlow (k) CD14⁺MC clusters. HLA-DRhi are shown in (i). Results are shown as mean fold change (±SEM).

FIG. 3: Surgery induces time-dependent and cell-type specific activation of immune signaling networks. (a) A heat map depicting hand-gated major immune cell subsets (rows, FIG. 7) and sampling times after surgery (columns). Within each block, changes in phosphorylation state of 11 intracellular signaling proteins (y-axis) are individually depicted for 26 patients (x-axis). The color scale indicates changes in phospho-signal median intensity (arcsinh ratio) compared to baseline. (b) Heat map depicting for each signaling protein, cell subset, and time point whether phosphorylation signals significantly increased (yellow, q<0.01, SAM Two class paired), decreased (blue, q<0.01), or remained unchanged (black, q>0.01). The color scale indicates mean fold-change of the signaling responses compared to baseline. Signaling responses in CD14+MCs and CD4+ T cells were most prominent (red). (c) Pearson correlation coefficients between changes in phosphorylation states of 11 signaling proteins in CD14+MCs at 1 h, 24 h, and 72 h after surgery were determined. Correlations within each (solid lines) and across (dash lines) time point(s) are depicted as black (|R|>0.7) and gray lines (|R|>0.5). (d) Signaling modules in CD14⁺MCs at 1 h, 24 h, and 72 h were identified by cutting the dendrograms of clustered correlation coefficients (FIG. 13) using a threshold of R>0.7. (e) At 72 h, module 1 split into modules 1a (pNF-κB, prpS6, pCREB) and 1b (pSTAT1) that correlated with each other (R=0.46, red line). At 24 h, module 2 split into modules 2a (pMAPKAP2, pP38) and 2b (pERK, pP90RSK) that correlated with each other (R=0.45, red line).

FIG. 4. Functional recovery and resolution of pain after surgery vary greatly among patients. (a-c). Heat maps depict the recovery parameters (a) global functioning, (b) hip function, and (c) pain for individual patients over the 6-week observation period. Global functioning was assessed with the Surgical Recovery Scale (SRS; 0-100=worst-best function). Pain and impairment of hip function were assessed with adapted versions of the Western Ontario and McMaster Universities Arthritis Index (WOMAC, pain 0-40=no pain-worst imaginable pain; function 0-60=no impairment-severe functional impairment). The heat maps reflect significant variability for extent and rate of recovery across all three outcome domains. (d-f). Box plots depict medians and interquartile ranges of (d) SRS, (e) WOMAC function, and (f) WOMAC pain scores (bars indicate 10th and 90th percentiles). An inset graph in panel f depicts the median daily analgesic consumption expressed as the dose equivalent of intravenous hydromorphone. Graphical information regarding pain and analgesic consumption are jointly presented, as these variables are inter-dependent. (g-i). Clinical recovery parameters were derived to quantify rate of recovery for the three outcomes. Derived parameters were (g) time to regain 50% of global functioning, (h) time to mild functional impairment of the hip, and (i) time to mild pain. Bars indicate median and interquartile range; open circles indicate individual data points.

FIG. 5. STAT3, CREB, and NF-κB signaling in CD14⁺MC subsets strongly correlate with surgical recovery. (a) CD45⁺CD66− cells obtained at BL and at 1 h, 24 h, and 72 h after surgery were clustered using an unsupervised approach (panels 1 and 2, FIG. 2b). Immune features, which include frequencies and signaling responses of 11 phospho-proteins, were derived for every cluster (panel 3). SAM Quantitative was used to detect significant correlations between immune features and parameters of clinical recovery (q<0.01, panel 4). Cell cluster phenotypes were identified using cell surface marker expression (panel 5). (b) Significant correlations were obtained for STAT3 signaling in cluster A (left panel), CREB signaling in cluster B (middle panel), and NF-κB signaling in cluster C (right panel) with recovery of global functioning, function of the hip, and resolution of pain. Clusters A and B were CD14⁺HLA-DR^{low} MCs; cluster C was CD14⁺HLA-DR^{hi} MCs (FIG. 14, 15). (c) Cells were hand-gated using 12 surface markers (blue line). Representative 2D plots are shown for one patient at 24 h (upper panel) and 1 h (middle and lower panels) after surgery. Percent cells in parent gate are shown. Cells contained in Clusters A, B, or C (blue shadow) are overlaid onto the entire cell population (gray). (d) Significant correlations between signaling responses and parameters of clinical recovery identified using an unsupervised approach were replicated with hand-gated data. Depicted are regression lines and 95% confidence intervals (solid and dashed lines), Spearman's ranked correlation coefficients, false discovery rates (q), and p-values.

FIG. 6. Assay performance and validation. (a) Cell population frequencies were measured in triplicate in whole blood obtained from one patient 1 h before and 1 h and 24 h after surgery. Single-cell data from the samples were manually gated into 13 cell populations based on the expression of 21 surface markers and DNA content (FIG. 7). Results are shown for six major immune cell subsets. Granulocyte frequency (left) is represented as percent total of CD235⁻CD61⁻ leukocytes. The frequency of all other cell types (right) is represented as percent total of CD235⁻CD61⁻CD45⁺CD66⁻ cells. The median coefficient of variation across triplicates was 4% with an interquartile range of 2%-12%. Results are shown as means±SEMs of triplicate experiments. G: granulocytes, MC: CD14+ monocytes, NK: Natural killer, B: B cells, CD4: CD4⁺ T cells, CD8: CD8⁺ T cells. (b) Signaling responses in four immune cell subsets were quantified in a blood sample obtained 1 h before surgery. Four aliquots of the sample were treated with PBS (control), interleukin cocktail (100 ng/mL IL-2, 100 ng/mL IL-6, 20 ng/mL IFNγ, 2 ng/mL GMCSF), with 80 nM phorbol 12-myristate 13-acetate and 1.3 μM ionomycin (Pma/Iono), or with 0.5 mM sodium pervanadate (PVO4). The heatmap shows phosphorylation changes of seven intracellular signaling molecules for the four immune cell subsets. The color scale indicates differences in median intensity (arcsinh ratio) between each of the three stimulation conditions and the control condition (PBS). The three stimulation conditions evoked expected signaling patterns. For example, in B cells the combined stimulation with 3 IL-6, IL-2, IFN<, and GM-CSF resulted in phosphorylation of STAT 1, 3, and 5 (arcsinh ratios 0.73, 0.6, and 1.46), but did not result in phosphorylation of ERK1/2 or CREB (arcsinh ratio 0.01, and −0.01). Similarly, in CD4+ T cells sodium pervanadate induced phosphorylation of ERK1/2 and CREB (arcsinh ratios 1.57 and 1.32). All phospho-specific antibodies used in this study were validated in a similar fashion (data not shown). (c) Phosphorylation levels of seven intracellular signaling molecules were quantified in triplicate for six immune cell subsets in blood samples obtained from one patient 1 h before and 1 h and 24 h after surgery. Phosphorylation changes in response to surgery were calculated for each signaling molecule as the difference of inverse hyperbolic sine medians between baseline and 1 h and 24 h after surgery (arcsinh ratio). Six signaling molecules (pSTAT1, pSTAT3, pSTAT5, pCREB, pP38, prpS6) showed statistically significant changes 1 h or 24 h after surgery. Significant changes were reproducible across triplicates as indicated by a median coefficient of variation of 24% with an interquartile range of 15-33%. Results are shown as means±SEMs. Statistical significance was inferred if confidence intervals did not include zero (*95%, **99%).

FIG. 7. Manual gating strategy. Gating strategy to define major immune cell types. Data are from a representative sample. Gates and plots were generated using cytobank.org. EM: effector memory, CM: central memory, NK: natural killer, pDC: plasmacytoid dendritic cell, cDC: classical dendritic cell.

FIG. 8. Changes in cell frequencies in serial samples from the six patients included in the pilot study. Surgery-induced changes in cell frequencies are shown for the pilot study of six patients. The relative size of cell compartments was quantified for neutrophils, CD14⁺ monocytes (MC), and CD4⁺ and CD8⁺ T cells. Samples were obtained 1 h before and 1 h, 24 h, 72 h, and 6 weeks (6 wks) after surgery. Neutrophils are expressed as percent of total hematopoietic cells (CD61⁻CD235⁻), whereas CD14⁺MCs and T cells are expressed as percent total of mononuclear cells (CD45⁺CD66⁻). Depicted are mean fold-changes±SEMs. A false discovery rate <0.01 (**) indicates statistical significance.

FIG. 9. Consort chart. Two hundred and fifty-one patients were assessed for eligibility, 50 were consented, 39 underwent total hip arthroplasty under the approved protocol, and 32 completed the study. Six patients were included in the pilot study, and 26 patients were included in the main study.

FIG. 10. Annotation of cluster hierarchy plots based on surface marker expression. Unsupervised hierarchical clustering produced a branching structure that allowed grouping CD45⁺CD66⁻ cells into known immune cell compartments. Mass cytometry data measured in samples from 26 patients 1 h before (BL) and 1 h, 24 h and 72 h after surgery was clustered together using the expression levels of 21 surface markers. Cell surface antibodies used for the clustering were CD7, CD19, CD11b, CD4, CD8, CD127, CCR7, CD123, CD45RA, CD33, CD11c, CD14, CD16, FoxP3, CD25, CD3, HLA-DR, and CD56. Upper panels: Coloring clusters based on cell surface marker expression highlights compartments for CD14⁺MCs, cDCs, pDCs, NK cells, B cells, CD4+ T cells, and CD8⁺ T cells. Lower panels: Cell frequencies within clusters corresponding to each immune compartment are depicted at 1 h, 24 h and 72 h after surgery. Changes in immune cell distribution within these clusters are similar to changes observed for immune cell compartments identified with a conventional gating strategy (FIG. 2a). Results represent mean fold changes (±SEM) in 26 patients.

FIG. 11. SAM analysis of cell frequency changes across clusters. (a) CD45⁺CD66⁻ cell cluster plot. Major immune compartments are depicted by contours. (b) Significant changes in cell frequency 1 h, 24 h or 72 h after surgery were determined with SAM Multiclass for each cluster. Cell frequencies increased in 29 clusters (shaded in red, q<0.01), increased then decreased in 14 clusters (shaded in gray, q<0.01), decreased in 19 clusters (shaded in blue, q<0.01), or remained unchanged in 107 clusters. Changes are shown across all time points. Clusters within the CD14⁺MCs compartment expanded the most (mean fold-change 4.0±0.28).

FIG. 12. Signaling responses over time in innate and adaptive immune compartments. (a) Depicted are phospho-signals for pSTAT3 in CD14⁺MCs, CD4⁺ Tcells and CD8⁺ Tcells. (b) Biphasic signaling responses in CD14⁺MCs were observed in phospho-signals for prpS6, pCREB and pNF-κB, pERK, pP38, pMAPKAPK2, and pP90RSK in CD14⁺MCs. Signaling responses are represented as changes over baseline phosphorylation status (arcsinh ratio over BL). Circles represent individual patients. Results are shown as mean differences (±SEM) from baseline. False discovery rate q<0.01 (**) indicate statistical significance (SAM Two class paired).

FIG. 13. Correlation heat maps and module derivation in CD14+ monocytes. Maps visualize the strength of the correlation between signaling responses in CD14+MCs at 1 h, 24 h and 72 h after surgery. Clustering signaling responses based on correlation coefficient revealed four modules that appeared at each time point (FIG. 3d, e). Module 1: pNF-κB (P65), prpS6, pCREB, and pSTAT1. Module 2: pMAPKAPK2 (MK2), pP38, pERK, and pP90RSK. Module 3: pSTAT5 and pPLCγ2. Module 4: pSTAT3. The color scale indicates correlation strength analyzed using Pearson's correlation coefficient (R).

FIG. 14. Immune feature correlations and identification of clusters A1, A2, and A3. (a-c) STAT3 signaling response between 1 h and 24 h in CD14+MC clusters. Significant and strong correlations were detected between STAT3 signaling in three cell clusters (A1, A2, A3) and the time to regain 50% of global functioning (q<0.01, SAM Quantitative). (d) The histograms shown in the top row serve as a reference and depict the expression of 18 out of 21 surface markers used to identify monocytes in all clusters. The bottom four rows display results for the cell clusters A, A1, A2, A3 identifying them as monocytes with low or moderate HLA-DR expression.

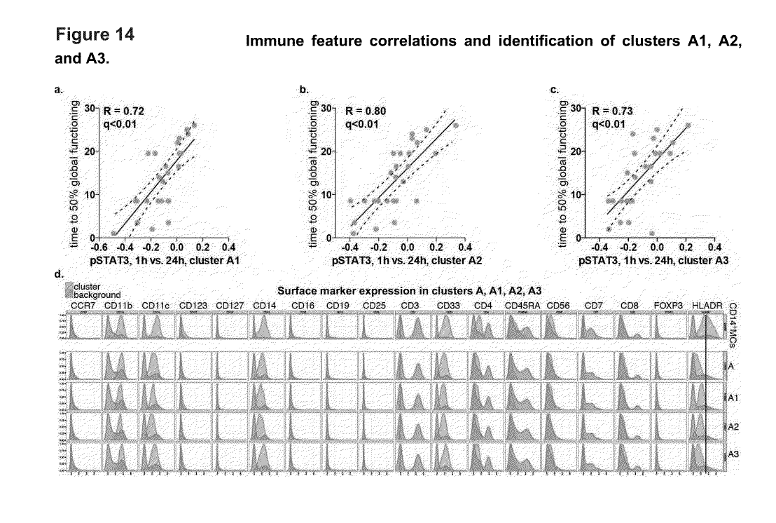

FIG. 15. Identification of Clusters B and C. The histograms shown in the top row serve as a reference and depict the expression of 18 out of 21 surface markers used to identify monocytes in all clusters. The bottom two rows display results for cell clusters B and C identifying them as monocytes with low (cluster B) or high (cluster C) HLA-DR expression.

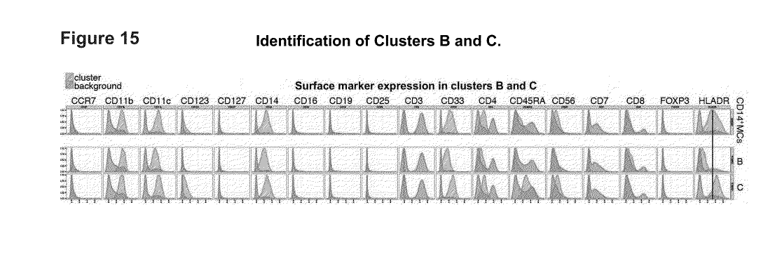

FIG. 16. Ex vivo response to LPS. pMAPKAPK2 signaling response between the untreated baseline sample and a baseline sample treated with 1 μg/ml of LPS in CD14+MC clusters. Significant and strong correlations were detected between pMAPKAPK2 and in 13 cell clusters and the time to mild functional impairment of the hip (R=0.63-0.70, q<0.01, SAM Quantitative). These clusters were identified as having a CD14+MC phenotype.

### DETAILED DESCRIPTION

These and other features of the present teachings will become more apparent from the description herein. While the present teachings are described in conjunction with various embodiments, it is not intended that the present teachings be limited to such embodiments. On the contrary, the present teachings encompass various alternatives, modifications, and equivalents, as will be appreciated by those of skill in the art.

Most of the words used in this specification have the meaning that would be attributed to those words by one skilled in the art. Words specifically defined in the specification have the meaning provided in the context of the present teachings as a whole, and as are typically understood by those skilled in the art. In the event that a conflict arises between an art-understood definition of a word or phrase and a definition of the word or phrase as specifically taught in this specification, the specification shall control.

It must be noted that, as used in the specification and the appended claims, the singular forms “a,” “an,” and “the” include plural referents unless the context clearly dictates otherwise.

Compositions and methods are provided for prognostic classification of patients following surgery according to their time to recovery, using an analysis at the single cell level of activation of signaling pathways in specific immune cell subsets. Patterns of response are obtained by quantitating specific activated signal proteins in immune cell subsets of interest, for a period of time following surgery. The pattern of response is indicative of the patient's response to surgical trauma, and the patient's time to recovery. Once a classification or prognosis has been made, it can be provided to a patient or caregiver. The classification can provide prognostic information to guide clinical decision making, both in terms of institution of and escalation of treatment, and in some cases may further include selection of a therapeutic agent or regimen.

The information obtained from the signaling protein patterns of response can be used to (a) determine type and level of therapeutic intervention warranted and (b) to optimize the selection of therapeutic agents. With this approach, therapeutic regimens can be individualized and tailored according to the response of the individual to surgery, thereby providing a regimen that is individually appropriate.

The terms “subject,” “individual,” and “patient” are used interchangeably herein to refer to a vertebrate, preferably a mammal, more preferably a human. Mammalian species that provide samples for analysis include canines; felines; equines; bovines; ovines; etc. and primates, particularly humans. Animal models, particularly small mammals, e.g. murine, lagomorpha, etc. can be used for experimental investigations. The methods of the invention can be applied for veterinary purposes, e.g. to determine the probable time to recover from surgery for a cat, dog, horse, etc. for use in decision-making whether to undergo surgery.

A “surgical trauma” as used herein means surgical procedures on the gastrointestinal tract, skeletal, vascular system, etc. Such surgical procedures include administration of anesthesia, large incisions to access the tissue being operated on, and the like. Common surgical procedures that may benefit from the methods of the invention include, without limitation, organ transplant; orthopedic surgery, e.g. partial or total hip replacement, partial or total knee replacement, etc.; cardiac or cardiothoracic surgery, e.g. coronary artery bypass, carotid endarterectomy, transplantation and heart failure surgery, oesophageal surgery and congenital surgery in adults and children; general surgery, e.g. appendectomy, cholecystectomy, mastectomy, partial colectomy, prostatectomy, tonsillectomy, etc. gynecologic surgery, e.g. Cesarean section, hysterectomy, etc., neurosurgery; plastic surgery; etc.

The stress response is the name given to the hormonal and metabolic changes which follow injury or trauma. This is part of the systemic reaction to injury which encompasses a wide range of endocrinological, immunological and hematological effects.

Cytokines have a major role in the inflammatory response to surgery and trauma. They have local effects of mediating and maintaining the inflammatory response to tissue injury, and also initiate some of the systemic changes which occur. After major surgery, the sentinal cytokines released include interleukin-1 (IL-1), tumor necrosis factor-α (TNF-α) and IL-6. An early response is the release of IL-1 and TNF-α from neutrophils and activated macrophages in the damaged tissues. These cytokines are also released from local tissue, including cells such as e.g. keratinocytes. This stimulates the production and release of more cytokines, in particular, IL-6, one of the main cytokines responsible for inducing the systemic changes known as the acute phase response.

A number of changes occur following tissue injury which are stimulated by cytokines, particularly IL-6. This is known as the ‘acute phase response’; one of its features is the production in the liver of acute phase proteins. These proteins act as inflammatory mediators, anti-proteinases and scavengers, and in tissue repair. They include C-reactive protein (CRP), fibrinogen, α₂₋macroglobulin and other anti-proteinases. The increase in serum concentrations of CRP follows the changes in IL-6. Production of other proteins in the liver, for example, albumin and transferrin, decreases during the acute phase response. Concentrations of circulating cations such as zinc and iron decrease, partly as a consequence of the changes in the production of the transport proteins.

There has been a great deal of interest in the modification of the stress response with respect to the potential beneficial effects on surgical outcome. Many factors other than analgesic regimens influence recovery from major surgery and the ability of the patient to return home and resume work. Behavioral and subjective changes are part of the response to surgery. Feelings of malaise and postoperative fatigue have a strong influence on recovery from surgery and return to work. Postoperative fatigue may encompass psychological and cultural mechanisms as well as physiological changes. Postoperative fatigue is a complex multifactorial issue.

Time to Recovery.

As is suggested above, the time to recovery after surgery is a complex phenomenon. The data provided herein demonstrate that different parameters for time to recovery can be correlated with specific responses of immune cell subsets. Parameters may be selected depending on the surgery, for example to recovery may be longer for difficult, complex procedures relative to less complex procedures. Parameters of general applicability may include time to regain 50% of global functioning; time to mild functional impairment of the affected tissue; and time to mild pain. The average time to recovery for a surgery of interest, or for a surgery of interest as performed in a specific setting, can be readily determined by one of skill in the art, and patients classified accordingly.

“Impaired global functioning” refers to the functional consequences of postoperative fatigue on regular daily activities, such as reading, etc. “Functional impairment” refers to function associated with the body part that was exposed to surgery, e.g. function of the hip after arthroplasty.

The WOMAC (Western Ontario and McMaster Universities) Index of Osteoarthritis. The WOMAC (Western Ontario and McMaster Universities) index is used to assess patients with osteoarthritis of the hip or knee using 24 parameters. It can be used to monitor the course of the disease or to determine the effectiveness of anti-rheumatic medications. See, for example, Bellamy et al. (1988) J Rheumatol. 15:1833-1840; and Stucki et al. (1998) Osteoarthritis and Cartilage 6: 79-86.

As used herein, the term “theranosis” refers to the use of results obtained from a diagnostic method to direct the selection of, maintenance of, or changes to a therapeutic regimen, including but not limited to the choice of one or more therapeutic agents, changes in dose level, changes in dose schedule, changes in mode of administration, and changes in formulation. Diagnostic methods used to inform a theranosis can include any that provides information on the state of a disease, condition, or symptom.

The terms “therapeutic agent”, “therapeutic capable agent” or “treatment agent” are used interchangeably and refer to a molecule or compound that confers some beneficial effect upon administration to a subject. The beneficial effect includes enablement of diagnostic determinations; amelioration of a disease, symptom, disorder, or pathological condition; reducing or preventing the onset of a disease, symptom, disorder or condition; and generally counteracting a disease, symptom, disorder or pathological condition.

As used herein, “treatment” or “treating,” or “palliating” or “ameliorating” are used interchangeably. These terms refer to an approach for obtaining beneficial or desired results including but not limited to a therapeutic benefit and/or a prophylactic benefit. By therapeutic benefit is meant any therapeutically relevant improvement in or effect on one or more diseases, conditions, or symptoms under treatment. For prophylactic benefit, the compositions may be administered to a subject at risk of developing a particular disease, condition, or symptom, or to a subject reporting one or more of the physiological symptoms of a disease, even though the disease, condition, or symptom may not have yet been manifested.

The term “effective amount” or “therapeutically effective amount” refers to the amount of an agent that is sufficient to effect beneficial or desired results. The therapeutically effective amount will vary depending upon the subject and disease condition being treated, the weight and age of the subject, the severity of the disease condition, the manner of administration and the like, which can readily be determined by one of ordinary skill in the art. The term also applies to a dose that will provide an image for detection by any one of the imaging methods described herein. The specific dose will vary depending on the particular agent chosen, the dosing regimen to be followed, whether it is administered in combination with other compounds, timing of administration, the tissue to be imaged, and the physical delivery system in which it is carried.

“Suitable conditions” shall have a meaning dependent on the context in which this term is used. That is, when used in connection with an antibody, the term shall mean conditions that permit an antibody to bind to its corresponding antigen. When used in connection with contacting an agent to a cell, this term shall mean conditions that permit an agent capable of doing so to enter a cell and perform its intended function. In one embodiment, the term “suitable conditions” as used herein means physiological conditions.

The term “inflammatory” response is the development of a humoral (antibody mediated) and/or a cellular response, which cellular response may be mediated by antigen-specific T cells or their secretion products) response by PAMPs and DAMPs, and innate immune cells. An “immunogen” is capable of inducing an immunological response against itself on administration to a mammal or due to autoimmune disease.

The terms “biomarker,” “biomarkers,” “marker” or “markers” for the purposes of the invention refer to, without limitation, proteins together with their related metabolites, mutations, variants, polymorphisms, modifications, fragments, subunits, degradation products, elements, and other analytes or sample-derived measures. Markers can include expression levels of an intracellular protein, e.g. I-κB protein level, or extracellular protein (e.g. HLA-DR). Markers particularly include activated proteins, for example where a marker may be the active, phosphorylated form of a protein involved in cellular signaling pathways, e.g. pSTAT1, pSTAT3, pSTAT5, pNF-κB, pCREB, and the like. Markers include, without limitation, the antigens recognized by any one of the antibodies set forth in Table 2. Markers can also include combinations of any one or more of the foregoing measurements, including temporal trends and differences. Broadly used, a marker can also refer to an immune cell subset, e.g. the presence of elevated numbers of CD14⁺ monocytes.

To “analyze” includes determining a set of values associated with a sample by measurement of a marker (such as, e.g., presence or absence of a marker or constituent expression levels) in the sample and comparing the measurement against measurement in a sample or set of samples from the same subject or other control subject(s). The markers of the present teachings can be analyzed by any of various conventional methods known in the art. To “analyze” can include performing a statistical analysis, e.g. normalization of data, determination of statistical significance, determination of statistical correlations, clustering algorithms, and the like.

A “sample” in the context of the present teachings refers to any biological sample that is isolated from a subject, generally a sample comprising circulating immune cells. A sample can include, without limitation, an aliquot of body fluid, whole blood, PBMC (white blood cells or leucocytes), tissue biopsies, synovial fluid, lymphatic fluid, ascites fluid, and interstitial or extracellular fluid. “Blood sample” can refer to whole blood or a fraction thereof, including blood cells, white blood cells or leucocytes. Samples can be obtained from a subject by means including but not limited to venipuncture, biopsy, needle aspirate, lavage, scraping, surgical incision, or intervention or other means known in the art.

Ex vivo activation of a sample, for the purposes of the present invention, refers to the contacting of a sample, e.g. a blood sample or cells derived therefrom, outside of the body with an stimulating agent. In some embodiments whole blood is preferred. The sample may be diluted or suspended in a suitable medium that maintains the viability of the cells, e.g. minimal media, PBS, etc. The sample can be fresh or frozen.

Stimulating agents of interest are those agents that activate CD14⁺ monocytes. Without limiting the invention, it is believed that the activation mimics the effect of surgery, and thus provides an in vitro correlate for the effects of surgery. In some embodiments, the activation agent is a TLR agonist, including without limitation activators of TLR2 and TLR4. Generally the activation of cells ex vivo is compared to a negative control, e.g. medium only, or an agent that does not elicit activation.

TLRs are evolutionarily conserved receptors important for defense against microbial infection. TLRs recognize highly conserved structural motifs known as pathogen-associated microbial patterns (PAMPs), which are exclusively expressed by microbial pathogens, or danger-associated molecular patterns (DAMPs) that are endogenous molecules released from necrotic or dying cells. PAMPs include various bacterial cell wall components such as lipopolysaccharide (LPS), peptidoglycan (PGN) and lipopeptides, as well as flagellin, bacterial DNA and viral double-stranded RNA. DAMPs include intracellular proteins such as heat shock proteins as well as protein fragments from the extracellular matrix. Stimulation of TLRs by the corresponding PAMPs or DAMPs initiates signaling cascades leading to the activation of transcription factors, such as AP-1, NF-κB and interferon regulatory factors (IRFs). Signaling by TLRs result in a variety of cellular responses including the production of interferons (IFNs), pro-inflammatory cytokines and effector cytokines that direct the adaptive immune response. Ten human and twelve murine TLRs have been characterized, TLR1 to TLR10 in humans, and TLR1 to TLR9, TLR11, TLR12 and TLR13 in mice, the homolog of TLR10 being a pseudogene.

TLR2 is essential for the recognition of a variety of PAMPs from Gram-positive bacteria, including bacterial lipoproteins, lipomannans and lipoteichoic acids. TLR3 is implicated in virus-derived double-stranded RNA. TLR4 is predominantly activated by lipopolysaccharide. TLR5 detects bacterial flagellin and TLR9 is required for response to unmethylated CpG DNA. TLR7 and TLR8 recognize small synthetic antiviral molecules, and single-stranded RNA. TLR11 has been reported to recognize uropathogenic E. coli and a profilin-like protein from Toxoplasma gondii. The repertoire of specificities of the TLRs is extended by the ability of TLRs to heterodimerize with one another. For example, dimers of TLR2 and TLR6 are required for responses to diacylated lipoproteins while TLR2 and TLR1 interact to recognize triacylated lipoproteins. Specificities of the TLRs are also influenced by various adapter and accessory molecules, such as MD-2 and CD14 that form a complex with TLR4 in response to LPS.

Agonists of TLRs include, without limitation, TLR1+ TLR2: triacylated lipoproteins (pam3csk4), peptidoglycans, lipopolysaccharides; TLR2+ TLR6: diacylated lipoproteins (fsl-1); heat shock proteins (hsp 60, 70, gp96); high mobility group proteins (hmgb1); proteoglycans (versican, hyaluronic acid fragments); TLR3: dsRNA (poly (i:c)); tRNA; siRNA; mRNA; TLR4: lipopolysaccharides (LPS); paclitaxel; heat shock proteins (hsp22, 60, 70, 72, gp96); high mobility group proteins (hmgb1); proteoglycans (versican, heparin sulfate, hyaluronic acid fragments); fibronectin, tenascin-c; TLR5: flagellin; TLR7: ssRNA; imidazoquinolines (r848); guanosine analogs (loxoribine); TLR8: ssRNA, imidazoquinolines (r848); TLR9: cpg DNA and oligonucleotides; chromatin IgG complex; TLR10: profilin-like proteins. Lipopolysaccharides are major components of the outer membrane of Gram-negative bacteria. In blood, LPS binds to LPS-binding protein (LBP), which circulates in the bloodstream where it recognizes and forms a high-affinity complex with the lipid A moiety of LPS and then forms a ternary complex with CD14, thus enabling LPS to be transferred to the LPS receptor complex comprising TLR4.

In some embodiments of the invention the activator is an LPS, which can be added to a patient sample or medium comprising cells from a patient sample in a dose effective to activate CD14+ monocytes, e.g. at a concentration of at least about 1 ng/ml, at least about 10 ng/ml, at least about 100 ng/ml, at least about 1 μg/ml and not more than about 100 μg/ml, where the concentration may be from about 0.1 to about 10 μg/ml. A dose response curve is readily performed by one of skill in the art to optimize response from the cells. Where the stimulating agent is other than LPS, the dose may be equivalent to the response seen with LPS from about 0.1 to about 10 μg/ml.

The cells are incubated for a period of time sufficient for activation. For example, where the stimulating agent is LPS, the time for action can be up to about 1 hour, up to about 45 minutes, up to about 30 minutes, up to about 15 minutes, and may be up to about 10 minutes or up to about 5 minutes. Following activation, the cells are fixed for analysis. In other embodiments the period of time may be up to about 24 hours.

A “dataset” is a set of numerical values resulting from evaluation of a sample (or population of samples) under a desired condition. The values of the dataset can be obtained, for example, by experimentally obtaining measures from a sample and constructing a dataset from these measurements; or alternatively, by obtaining a dataset from a service provider such as a laboratory, or from a database or a server on which the dataset has been stored. Similarly, the term “obtaining a dataset associated with a sample” encompasses obtaining a set of data determined from at least one sample. Obtaining a dataset encompasses obtaining a sample, and processing the sample to experimentally determine the data, e.g., via measuring antibody binding, or other methods of quantitating a signaling response. The phrase also encompasses receiving a set of data, e.g., from a third party that has processed the sample to experimentally determine the dataset.

“Measuring” or “measurement” in the context of the present teachings refers to determining the presence, absence, quantity, amount, or effective amount of a substance in a clinical or subject-derived sample, including the presence, absence, or concentration levels of such substances, and/or evaluating the values or categorization of a subject's clinical parameters based on a control, e.g. baseline levels of the marker.

Classification can be made according to predictive modeling methods that set a threshold for determining the probability that a sample belongs to a given class. The probability preferably is at least 50%, or at least 60% or at least 70% or at least 80% or higher. Classifications also can be made by determining whether a comparison between an obtained dataset and a reference dataset yields a statistically significant difference. If so, then the sample from which the dataset was obtained is classified as not belonging to the reference dataset class. Conversely, if such a comparison is not statistically significantly different from the reference dataset, then the sample from which the dataset was obtained is classified as belonging to the reference dataset class.

The predictive ability of a model can be evaluated according to its ability to provide a quality metric, e.g. AUC or accuracy, of a particular value, or range of values. In some embodiments, a desired quality threshold is a predictive model that will classify a sample with an accuracy of at least about 0.7, at least about 0.75, at least about 0.8, at least about 0.85, at least about 0.9, at least about 0.95, or higher. As an alternative measure, a desired quality threshold can refer to a predictive model that will classify a sample with an AUC (area under the curve) of at least about 0.7, at least about 0.75, at least about 0.8, at least about 0.85, at least about 0.9, or higher.

As is known in the art, the relative sensitivity and specificity of a predictive model can be “tuned” to favor either the selectivity metric or the sensitivity metric, where the two metrics have an inverse relationship. The limits in a model as described above can be adjusted to provide a selected sensitivity or specificity level, depending on the particular requirements of the test being performed. One or both of sensitivity and specificity can be at least about at least about 0.7, at least about 0.75, at least about 0.8, at least about 0.85, at least about 0.9, or higher.

“Affinity reagent”, or “specific binding member” may be used to refer to a affinity reagent, such as an antibody, ligand, etc. that selectively binds to a protein or marker of the invention. The term “affinity reagent” includes any molecule, e.g., peptide, nucleic acid, small organic molecule. For some purposes, an affinity reagent selectively binds to a cell surface marker, e.g. CD3, CD14, CD66, HLA-DR, CD11b, CD33, CD45, CD235, CD61, CD19, CD4, CD8, CD123, CCR7, and the like. For other purposes an affinity reagent selectively binds to a cellular signaling protein, particularly one which is capable of detecting an activation state of a signaling protein over another activation state of the signaling protein. Signaling proteins of interest include, without limitation, pSTAT3, pSTAT1, pCREB, pSTAT6, pPLCγ2, pSTAT5, pSTAT4, pERK, pP38, prpS6, pNF-κB (p65), pMAPKAPK2, pP90RSK, etc.

In some embodiments, the affinity reagent is a peptide, polypeptide, oligopeptide or a protein, particularly antibodies and specific binding fragments and variants thereof. The peptide, polypeptide, oligopeptide or protein can be made up of naturally occurring amino acids and peptide bonds, or synthetic peptidomimetic structures. Thus “amino acid”, or “peptide residue”, as used herein include both naturally occurring and synthetic amino acids. Proteins including non-naturally occurring amino acids can be synthesized or in some cases, made recombinantly; see van Hest et al., FEBS Lett 428:(I-2) 68-70 May 22, 1998 and Tang et al., Abstr. Pap Am. Chem. S218: U138 Part 2 Aug. 22, 1999, both of which are expressly incorporated by reference herein.

Methods of the present invention can be used to detect any particular signaling protein in a sample that is antigenically detectable and antigenically distinguishable from other signaling proteins which are present in the sample. For example, activation state-specific antibodies can be used to identify distinct signaling cascades of a subset or subpopulation of complex cell populations; and the ordering of protein activation (e.g., kinase activation) in potential signaling hierarchies. Hence, in some embodiments the expression and phosphorylation of one or more polypeptides are detected and quantified using methods of the present invention. As used herein, the term “activation state-specific antibody” or “activation state antibody” or grammatical equivalents thereof, refer to an antibody that specifically binds to a corresponding and specific antigen. Preferably, the corresponding and specific antigen is a specific form of an signaling protein. Also preferably, the binding of the activation state-specific antibody is indicative of a specific activation state of a specific signaling protein.

The term “antibody” includes full length antibodies and antibody fragments, and can refer to a natural antibody from any organism, an engineered antibody, or an antibody generated recombinantly for experimental, therapeutic, or other purposes as further defined below. Examples of antibody fragments, as are known in the art, such as Fab, Fab′, F(ab′)2, Fv, scFv, or other antigen-binding subsequences of antibodies, either produced by the modification of whole antibodies or those synthesized de novo using recombinant DNA technologies. The term “antibody” comprises monoclonal and polyclonal antibodies. Antibodies can be antagonists, agonists, neutralizing, inhibitory, or stimulatory. They can be humanized, glycosylated, bound to solid supports, and possess other variations.

The antigenicity of an activated isoform of an signaling protein is distinguishable from the antigenicity of non-activated isoform of an signaling protein or from the antigenicity of an isoform of a different activation state. In some embodiments, an activated isoform of an element possesses an epitope that is absent in a non-activated isoform of an element, or vice versa. In some embodiments, this difference is due to covalent addition of moieties to an element, such as phosphate moieties, or due to a structural change in an element, as through protein cleavage, or due to an otherwise induced conformational change in an element which causes the element to present the same sequence in an antigenically distinguishable way. In some embodiments, such a conformational change causes an activated isoform of a signaling protein to present at least one epitope that is not present in a non-activated isoform, or to not present at least one epitope that is presented by a non-activated isoform of the element.

Many antibodies, many of which are commercially available (for example, see Cell Signaling Technology, www.cellsignal.com or Becton Dickinson, www.bd.com) have been produced which specifically bind to the phosphorylated isoform of a protein but do not specifically bind to a non-phosphorylated isoform of a protein. Many such antibodies have been produced for the study of signal transducing proteins which are reversibly phosphorylated. Particularly, many such antibodies have been produced which specifically bind to phosphorylated, activated isoforms of protein. Examples of proteins that can be analyzed with the methods described herein include, but are not limited to, NF-κB, CREB and STAT3.

The methods the invention may utilize affinity reagents comprising a label, labeling element, or tag. By label or labeling element is meant a molecule that can be directly (i.e., a primary label) or indirectly (i.e., a secondary label) detected; for example a label can be visualized and/or measured or otherwise identified so that its presence or absence can be known.

A compound can be directly or indirectly conjugated to a label which provides a detectable signal, e.g. non-radioactive isotopes, radioisotopes, fluorophores, enzymes, antibodies, particles such as magnetic particles, chemiluminescent molecules, molecules that can be detected by mass spec, or specific binding molecules, etc. Specific binding molecules include pairs, such as biotin and streptavidin, digoxin and anti-digoxin etc. Examples of labels include, but are not limited to, metal isotopes, optical fluorescent and chromogenic dyes including labels, label enzymes and radioisotopes. In some embodiments of the invention, these labels can be conjugated to the affinity reagents. In some embodiments, one or more affinity reagents are uniquely labeled.

Labels include optical labels such as fluorescent dyes or moieties. Fluorophores can be either “small molecule” fluors, or proteinaceous fluors (e.g. green fluorescent proteins and all variants thereof). In some embodiments, activation state-specific antibodies are labeled with quantum dots as disclosed by Chattopadhyay et al. (2006) Nat. Med. 12, 972-977. Quantum dot labeled antibodies can be used alone or they can be employed in conjunction with organic fluorochrome-conjugated antibodies to increase the total number of labels available. As the number of labeled antibodies increase so does the ability for subtyping known cell populations.

Activation state-specific antibodies can be labeled using chelated or caged lanthanides as disclosed by Erkki et al. (1988) J. Histochemistry Cytochemistry, 36:1449-1451, and U.S. Pat. No. 7,018,850. Other labels are tags suitable for Inductively Coupled Plasma Mass Spectrometer (ICP-MS) as disclosed in Tanner et al. (2007) Spectrochimica Acta Part B: Atomic Spectroscopy 62(3):188-195. Isotope labels suitable for mass cytometry may be used, for example as described in published application US 2012-0178183.

Alternatively, detection systems based on FRET can be used. FRET find use in the invention, for example, in detecting activation states that involve clustering or multimerization wherein the proximity of two FRET labels is altered due to activation. In some embodiments, at least two fluorescent labels are used which are members of a fluorescence resonance energy transfer (FRET) pair.

When using fluorescent labeled components in the methods and compositions of the present invention, it will recognized that different types of fluorescent monitoring systems, e.g., cytometric measurement device systems, can be used to practice the invention. In some embodiments, flow cytometric systems are used or systems dedicated to high throughput screening, e.g. 96 well or greater microtiter plates. Methods of performing assays on fluorescent materials are well known in the art and are described in, e.g., Lakowicz, J. R., Principles of Fluorescence Spectroscopy, New York: Plenum Press (1983); Herman, B., Resonance energy transfer microscopy, in: Fluorescence Microscopy of Living Cells in Culture, Part B, Methods in Cell Biology, vol. 30, ed. Taylor, D. L. & Wang, Y.-L., San Diego: Academic Press (1989), pp. 219-243; Turro, N. J., Modern Molecular Photochemistry, Menlo Park: Benjamin/Cummings Publishing Col, Inc. (1978), pp. 296-361.

The detecting, sorting, or isolating step of the methods of the present invention can entail fluorescence-activated cell sorting (FACS) techniques, where FACS is used to select cells from the population containing a particular surface marker, or the selection step can entail the use of magnetically responsive particles as retrievable supports for target cell capture and/or background removal. A variety of FACS systems are known in the art and can be used in the methods of the invention (see e.g., WO99/54494, filed Apr. 16, 1999; U.S. Ser. No. 20010006787, filed Jul. 5, 2001, each expressly incorporated herein by reference).

In some embodiments, a FACS cell sorter (e.g. a FACSVantage™ Cell Sorter, Becton Dickinson Immunocytometry Systems, San Jose, Calif.) is used to sort and collect cells based on their activation profile (positive cells) in the presence or absence of an increase in activation level in an signaling protein in response to a modulator. Other flow cytometers that are commercially available include the LSR II and the Canto II both available from Becton Dickinson. See Shapiro, Howard M., Practical Flow Cytometry, 4th Ed., John Wiley & Sons, Inc., 2003 for additional information on flow cytometers.

In some embodiments, the cells are first contacted with labeled activation state-specific affinity reagents (e.g. antibodies) directed against specific activation state of specific signaling proteins. In such an embodiment, the amount of bound affinity reagent on each cell can be measured by passing droplets containing the cells through the cell sorter. By imparting an electromagnetic charge to droplets containing the positive cells, the cells can be separated from other cells. The positively selected cells can then be harvested in sterile collection vessels. These cell-sorting procedures are described in detail, for example, in the FACSVantage™ Training Manual, with particular reference to sections 3-11 to 3-28 and 10-1 to 10-17, which is hereby incorporated by reference in its entirety. See the patents, applications and articles referred to, and incorporated above for detection systems.

In some embodiments, the activation level of an signaling protein is measured using Inductively Coupled Plasma Mass Spectrometer (ICP-MS). An affinity reagent that has been labeled with a specific element binds to a marker of interest. When the cell is introduced into the ICP, it is atomized and ionized. The elemental composition of the cell, including the labeled affinity reagent that is bound to the signaling protein, is measured. The presence and intensity of the signals corresponding to the labels on the affinity reagent indicates the level of the signaling protein on that cell (Tanner et al. Spectrochimica Acta Part B: Atomic Spectroscopy, 2007 March; 62(3):188-195).

Mass cytometry, e.g. as described in the Examples provided herein, finds use on analysis. Mass cytometry, or CyTOF (DVS Sciences), is a variation of flow cytometry in which antibodies are labeled with heavy metal ion tags rather than fluorochromes. Readout is by time-of-flight mass spectrometry. This allows for the combination of many more antibody specificities in a single samples, without significant spillover between channels. For example, see Bodenmiller at a. (2012) Nature Biotechnology 30:858-867.

STAT Signaling Pathways.

In mammals seven members of the STAT family (STAT1, STAT2, STAT3, STAT4, STAT5a, STAT5b and STATE) have been identified. JAKs contain two symmetrical kinase-like domains; the C-terminal JAK homology 1 (JH1) domain possesses tyrosine kinase function while the immediately adjacent JH2 domain is enzymatically inert but is believed to regulate the activity of JH1. There are four JAK family members: JAK1, JAK2, JAK3 and tyrosine kinase 2 (Tyk2). Expression is ubiquitous for JAK1, JAK2 and TYK2 but restricted to hematopoietic cells for JAK3.

STATs can be activated in a JAK-independent manner by src family kinase members and by oncogenic FLt3 ligand-ITD (Hayakawa and Naoe, Ann N Y Acad Sci. 2006 November; 1086:213-22; Choudhary et al. Activation mechanisms of STATS by oncogenic FLt3 ligand-ITD. Blood (2007) vol. 110 (1) pp. 370-4).

STAT3 is a member of the STAT protein family. In response to cytokines and growth factors, STAT family members are phosphorylated by receptor-associated kinases and then form homo- or heterodimers that translocate to the cell nucleus, where they act as transcription activators. STAT3 is activated through phosphorylation of tyrosine 705, in response to various cytokines and growth factors. STAT3 mediates the expression of a variety of genes in response to cell stimuli, and thus plays a key role in many cellular processes such as cell growth and apoptosis.

STAT3 has been shown to interact with: AR; ELP2; EP300; EGFR; HIF1A; JAK1; JUN; KHDRBS1; MTOR; MYOD1; NDUFA13; NF-κB1; NR3C1; NCOA1; PML; RAC1; RELA; RET; RPA2; STAT1; Src; and TRIP10.

CREB Signaling Pathway.

CREB (cAMP response element-binding protein) is a cellular transcription factor. It binds to cAMP response elements (CRE), thereby increasing or decreasing the transcription of the downstream genes. Genes whose transcription is regulated by CREB include: c-fos, BDNF, tyrosine hydroxylase, and many neuropeptides. When activated CREB protein forms a dimer and binds to the CRE region of DNA through a leucine zipper motif. The protein also has a magnesium ion that facilitates binding to DNA. Transcriptional activity of CREB requires phosphorylation of the protein on a serine residue at position 119.

CBP binds to the ser133 phosphorylated region of CREB via a domain called KIX. The phosphorylated domain of CREB was termed KID for kinase-inducible domain. The KID domain of CREB comprises amino acid residues 101 to 160. The KID undergoes a coil-to-helix folding transition upon binding to KIX, forming 2 alpha helices. The amphipathic helix alpha-B of KID interacts with a hydrophobic groove defined by helices alpha-1 and alpha-3 of KIX. The other KID helix, alpha-A, contacts a different face of the alpha-3 helix. The phosphate group of the critical phosphoserine residue of KID forms a hydrogen bond to the side chain of tyr658 of KIX.

NF-κB signaling pathways. NF-κB (nuclear factor kappa-light-chain-enhancer of activated B cells) is a protein complex that controls transcription of DNA. NF-κB is found in almost all animal cell types and is involved in cellular responses to stimuli such as stress, cytokines, free radicals, ultraviolet irradiation, oxidized LDL, and regulates immune responses.

There are five proteins in the mammalian NF-κB family: NF-κB1, NF-κB2, RelA, RelB and c-Rel. All proteins of the NF-κB family share a Rel homology domain in their N-terminus. A subfamily of NF-κB proteins, including RelA (p65), RelB, and c-Rel, have a transactivation domain in their C-termini. In contrast, the NF-κB1 and NF-κB2 proteins are synthesized as large precursors, p105, and p100, which undergo processing to generate the mature NF-κB subunits, p50 and p52, respectively. The p50 and p52 proteins have no intrinsic ability to activate transcription.

NF-κB is important in regulating cellular responses because it belongs to the category of “rapid-acting” primary transcription factors, i.e., transcription factors that are present in cells in an inactive state and do not require new protein synthesis in order to become activated (other members of this family include transcription factors such as c-Jun, STATs, and nuclear hormone receptors). Known inducers of NF-κB activity are highly variable and include reactive oxygen species (ROS), tumor necrosis factor alpha (TNFα), interleukin 1-beta (IL-1β), bacterial lipopolysaccharides (LPS), isoproterenol, cocaine, and ionizing radiation.

In unstimulated cells, the NF-κB dimers are sequestered in the cytoplasm by a family of inhibitors, called IκBs (Inhibitor of KB), which contain multiple ankyrin repeats, which mask the nuclear localization signals (NLS) of NF-κB proteins and keep them sequestered in an inactive state in the cytoplasm. In some embodiments, a marker of interest is the p65 phosporylation at serine 529.

Monocytes.

Cells of the monocyte lineage are important elements of immune defense because these cells can phagocytize foreign material, present Ag to T cells, and produce a host of cytokines, including TNF, IL-1, and IL-6. The cells of the monocyte lineage derive from myelomonocytic stem cells in bone marrow. They mature to monocytes and, as such, they go into blood followed by migration into tissue. In tissue these cells are referred to as macrophages, which differentiate into phenotypically and functionally distinct cell types like alveolar macrophages, osteoclasts, or microglia cells.

The present invention incorporates information disclosed in other applications and texts. The following patent and other publications are hereby incorporated by reference in their entireties: Alberts et al., The Molecular Biology of the Cell, 4th Ed., Garland Science, 2002; Vogelstein and Kinzler, The Genetic Basis of Human Cancer, 2d Ed., McGraw Hill, 2002; Michael, Biochemical Pathways, John Wiley and Sons, 1999; Weinberg, The Biology of Cancer, 2007; Immunobiology, Janeway et al. 7th Ed., Garland, and Leroith and Bondy, Growth Factors and Cytokines in Health and Disease, A Multi Volume Treatise, Volumes 1A and IB, Growth Factors, 1996.

Unless otherwise apparent from the context, all elements, steps or features of the invention can be used in any combination with other elements, steps or features.

General methods in molecular and cellular biochemistry can be found in such standard textbooks as Molecular Cloning: A Laboratory Manual, 3rd Ed. (Sambrook et al., Harbor Laboratory Press 2001); Short Protocols in Molecular Biology, 4th Ed. (Ausubel et al. eds., John Wiley & Sons 1999); Protein Methods (Bollag et al., John Wiley & Sons 1996); Nonviral Vectors for Gene Therapy (Wagner et al. eds., Academic Press 1999); Viral Vectors (Kaplift & Loewy eds., Academic Press 1995); Immunology Methods Manual (I. Lefkovits ed., Academic Press 1997); and Cell and Tissue Culture: Laboratory Procedures in Biotechnology (Doyle & Griffiths, John Wiley & Sons 1998). Reagents, cloning vectors, and kits for genetic manipulation referred to in this disclosure are available from commercial vendors such as BioRad, Stratagene, Invitrogen, Sigma-Aldrich, and ClonTech.

The invention has been described in terms of particular embodiments found or proposed by the present inventor to comprise preferred modes for the practice of the invention. It will be appreciated by those of skill in the art that, in light of the present disclosure, numerous modifications and changes can be made in the particular embodiments exemplified without departing from the intended scope of the invention. Due to biological functional equivalency considerations, changes can be made in protein structure without affecting the biological action in kind or amount. All such modifications are intended to be included within the scope of the appended claims.

The subject methods are used for prophylactic or therapeutic purposes. As used herein, the term “treating” is used to refer to both prevention of relapses, and treatment of pre-existing conditions. For example, the prevention of inflammatory disease can be accomplished by administration of the agent prior to development of a relapse. The treatment of ongoing disease, where the treatment stabilizes or improves the clinical symptoms of the patient, is of particular interest.

Relevant articles include Krutzik et al., Nature Chemical Biology 23: 132-42, 2007; Irish et al., FLt3 ligand Y591 duplication and Bcl-2 over expression are detected in acute myeloid leukemia cells with high levels of phosphorylated wild-type p53, Blood 109: 2589-96 2007; Irish et al. Mapping normal and cancer cell signaling networks: towards single-cell proteomics, Nature Rev. Cancer, 6: 146-55 2006; Irish et al., Single cell profiling of potentiated phospho-protein networks in cancer cells, Cell, Vol. 118, 1-20 Jul. 23, 2004; Schulz, K. R., et al., Single-cell phospho-protein analysis by flow cytometry, Curr Protoc Immunol, Chapter 8: Units 8.17.1-20, 2007; Krutzik, P. O., et al., Coordinate analysis of murine immune cell surface markers and intracellular phosphoproteins by flow cytometry, J Immunol. 2005 1754: 2357-65; Krutzik, P. O., et al., Characterization of the murine immunological signaling network with phosphospecific flow cytometry, J Immunol. 175: 2366-73, 2005; Stelzer et al. Use of Multiparameter Flow Cytometry and Immunophenotyping for the Diagnosis and Classification of Acute Myeloid Leukemia, Immunophenotyping, Wiley, 2000; and Krutzik, P. O. and Nolan, G. P., Intracellular phospho-protein staining techniques for flow cytometry: monitoring single cell signaling events, Cytometry A.55:61-70, 2005; Hanahan D., Weinberg, The Hallmarks of Cancer, Cell 100:57-70, 2000; Krutzik et al, High content single cell drug screening with phosphospecific flow cytometry, Nat Chem Biol. 4:132-42, 2008. Guiding principles of statistical analysis can be found in Begg C B. (1987). Biases in the assessment of diagnostic tests. Stat in Med. 6, 411-423.; Bossuyt, P. M., et al. (2003) Towards complete and accurate reporting of studies of diagnostic accuracy: the STARD initiative. Clinical Chemistry 49, 1-6 (also in Ann. Intern. Med., BMJ and Radiology in 2003); CDRH, FDA. (2003). Statistical Guidance on Reporting Results from Studies Evaluating Diagnostic Tests: Draft Guidance (March, 2003); Pepe M S. (2003). The Statistical Evaluation of Medical Tests for Classification and Prediction. Oxford Press; Zhou X-H, Obuchowski N A, McClish D K. (2002).

### Methods of the Invention

Multiparametric analysis, at a single cell level, of cellular biological samples obtained from an individual contemplating or undergoing surgery is used to obtain a determination of changes in immune cell subsets, which changes include, without limitation, altered activation states of proteins involved in signaling pathways. It is surprisingly found that shortly after surgery, or in response to ex vivo activation as described herein, changes occur in signaling pathways of these immune cells that are predictive of the potential recovery status of the individual. For example, multiparameter flow cytometry at the single cell level measures the activation status of multiple intracellular signaling proteins, as well as assigning activation states of these proteins to the varied cell sub-sets within a complex cell population. Flow cytometry includes, without limitations FACS, mass cytometry, and the like.

Protein phosphorylation is a critical post translational process in controlling many cell functions such as migration, apoptosis, proliferation and differentiation. Site specific phosphorylation of proteins can be detected, for example, by incubating cells with labeled phospho-specific antibodies using flow cytometry.

The sample can be any suitable type that allows for the analysis of one or more cells, preferably a blood sample. Samples can be obtained once or multiple times from an individual. Multiple samples can be obtained from different locations in the individual (e.g., blood samples, bone marrow samples and/or lymph node samples), at different times from the individual, or any combination thereof.

When samples are obtained as a series, e.g., a series of blood samples obtained after surgery, the samples can be obtained at fixed intervals, at intervals determined by the status of the most recent sample or samples or by other characteristics of the individual, or some combination thereof. For example, samples can be obtained at intervals of approximately 1, 2, 3, or 4 hours, at intervals of approximately 1, 2, 3, 4, 5, 6, 7, 8, 9, 10, or 11 days, or some combination thereof. It will be appreciated that an interval may not be exact, according to an individual's availability for sampling and the availability of sampling facilities, thus approximate intervals corresponding to an intended interval scheme are encompassed by the invention. Generally, the most easily obtained samples are fluid samples. In some embodiments the sample or samples is blood. Where activation is performed ex vivo, a single sample obtained prior to surgery can be sufficient.

One or more cells or cell types, or samples containing one or more cells or cell types, can be isolated from body samples. The cells can be separated from body samples by red cell lysis, centrifugation, elutriation, density gradient separation, apheresis, affinity selection, panning, FACS, centrifugation with Hypaque, solid supports (magnetic beads, beads in columns, or other surfaces) with attached antibodies, etc. By using antibodies specific for markers identified with particular cell types, a relatively homogeneous population of cells can be obtained. Alternatively, a heterogeneous cell population can be used, e.g. circulating peripheral blood mononuclear cells.

In some embodiments, a phenotypic profile of a population of cells is determined by measuring the activation level of a signaling protein. The methods and compositions of the invention can be employed to examine and profile the status of any signaling protein in a cellular pathway, or collections of such signaling proteins. Single or multiple distinct pathways can be profiled (sequentially or simultaneously), or subsets of signaling proteins within a single pathway or across multiple pathways can be examined (sequentially or simultaneously).

In some embodiments, the basis for classifying cells is that the distribution of activation levels for one or more specific signaling proteins will differ among different phenotypes. A certain activation level, or more typically a range of activation levels for one or more signaling proteins seen in a cell or a population of cells, is indicative that that cell or population of cells belongs to a distinctive phenotype. Other measurements, such as cellular levels (e.g., expression levels) of biomolecules that may not contain signaling proteins, can also be used to classify cells in addition to activation levels of signaling proteins; it will be appreciated that these levels also will follow a distribution. Thus, the activation level or levels of one or more signaling proteins, optionally in conjunction with the level of one or more biomolecules that may or may not contain signaling proteins, of a cell or a population of cells can be used to classify a cell or a population of cells into a class. It is understood that activation levels can exist as a distribution and that an activation level of a particular element used to classify a cell can be a particular point on the distribution but more typically can be a portion of the distribution. In addition to activation levels of intracellular signaling proteins, levels of intracellular or extracellular biomolecules, e.g., proteins, can be used alone or in combination with activation states of signaling proteins to classify cells. Further, additional cellular elements, e.g., biomolecules or molecular complexes such as RNA, DNA, carbohydrates, metabolites, and the like, can be used in conjunction with activation states or expression levels in the classification of cells encompassed here.

In one embodiment of the invention, a method is provided for classifying or prognosing the recovery status of an individual following surgery, the method comprising determining levels of at least one marker in a patient, or in an activated sample of cells from the patient; where the marker(s) is indicative of the activation status of at least one signaling protein in circulating immune cells. In some embodiments the circulating immune cells are CD14⁺ monocytes, which cells can be gated or selected on one or more markers as previously defined herein. The CD14+ monocytes may be an HLA-DR^{low} subset, or an HLA-DR^{high} subset. In some embodiments the circulating immune cells are CD4+ T cells, or a subset thereof. In some embodiments the immune cells are profiled according to expression of one or more of CD3, CD14, CD66, HLA-DR, CD11b, CD33, CD45, CD235, CD61, CD19, CD4, CD8, CD123, and CCR7. In some embodiments the immune cells are profiled according to expression of one or more of CD3, CD14, CD66, HLA-DR, and CD11b. The profile can be performed with 1, 2, 3, 4, or all 5 of the markers.

In some embodiments of the invention, different gating strategies can be used in order to analyze a specific cell population (e.g., only CD14⁺MC) in a sample of mixed cell population. These gating strategies can be based on the presence of one or more specific surface markers. The following gate can differentiate between dead cells and live cells and the subsequent gating of live cells classifies them into, e.g. myeloid blasts, monocytes and lymphocytes. A clear comparison can be carried out by using two-dimensional contour plot representations, two-dimensional dot plot representations, and/or histograms. In some embodiments the immune cells are profiled by binding to affinity reagents specific for CD3, CD14, CD66, HLA-DR, and CD11b. The profiling may gate on cells that are CD3⁻, CD14⁺, CD66⁻, CD11b⁺, Cd14⁺.

The immune cells are analyzed for the presence of an activated form of a signaling protein of interest. Signaling proteins of interest include, without limitation, pSTAT3, pSTAT1, pCREB, pSTAT6, pPLCγ2, pSTAT5, pSTAT4, pERK, pP38, prpS6, pNF-κB (p65), pMAPKAPK2, and pP90RSK.

In some embodiments, cellular levels of one or more of pMAPKAP2, pERK, pCREB, pNF-κB (p65), pSTAT1 and pSTAT3 are analyzed. In some embodiments, 1 2 or 3 of pCREB, pNF-κB (p65), and pSTAT3 are analyzed. In other embodiments, pMAPKAP2 is measured in immune cells stimulated ex vivo. In some embodiments the analysis is gated on monocytes. In some embodiments the analysis is gated on CD14⁺ monocytes, and may be gated on CD14⁺HLA-DR^{low} monocytes or CD14⁺HLA-DR^{high} monocytes. In other embodiments, one or both of pSTAT3 and pSTAT5 are analyzed in CD4⁺ circulating T cells. In other embodiments, one or both of pSTAT3 and pSTAT5 are analyzed in CD8⁺ circulating T cells.

To determine if a change is significant, e.g. whether immune cell response to ex vivo stimulation results in a low level of pMAPKAP2, the pMAPKAP2 signal in a patient's baseline sample can be compared to a reference scale from a cohort of patients with known recovery outcomes.

Samples may be obtained at one or more time points. Where a sample at a single time point is used, comparison is made to a reference “base line” level for the presence of the activated form of the signaling protein of interest, which may be obtained from a normal control, a pre-determined level obtained from one or a population of individuals, from a negative control for ex vivo activation, and the like.

Where multiple samples are obtained from an individual, one sample may provide a “base line”, or reference level for comparative purposes. Samples suitable for this purpose include, without limitation, pre-surgery samples; and samples obtained shortly after surgery, e.g. within about 15 minute, within about 30 minutes, within about 45 minutes, within about 1 hour, within about 1.5 hours, within about 2 hours.

Samples of interest for prognostic classification can include samples obtained prior to surgery (for ex vivo activation); shortly after surgery, e.g. within about 15 minutes, within about 30 minutes, within about 45 minutes, within about 1 hour, within about 1.5 hours, within about 2 hours. Samples of interest for prognostic classification can include samples obtained after surgery, e.g. within about 6 hours, within about 12 hours, within about 18 hours, within about 24 hours, within about 30 hours, within about 36 hours, within about 42 hours, within about 48 hours. Samples of interest for prognostic classification can also include samples obtained in the medium term after surgery, e.g. within about 2 days, within about 3 days, within about 4 days, within about 5 days.

In some specific embodiments, an increase in pSTAT3 levels in monocytes, e.g. CD14+ monocytes after about 18-30 hours, and may be around 24 hours following surgery, compared to a base line level immediately following surgery, is indicative that an individual will require a longer period of time to achieve recovery, as assessed, for example, by time to 50% global function, relative to an individual that does not show a significant increase in pSTAT3. Lack of at least mild decrease between 1 h and 24 h is also associated with delayed recovery.

In some specific embodiments, a decrease in pCREB levels in monocytes, e.g. CD14+ monocytes immediately following surgery, for example within about 15 minutes, within about 30 minutes, within about 45 minutes, within about 1 hour, within about 1.5 hours, within about 2 hours compared to a pre-surgery base line level, is indicative that an individual will require a shorter period of time to achieve recovery, as assessed, for example, by time to mild functional impairment, relative to an individual that does not show a significant decrease in pCREB. In other words, lower CREB and Nf-κB phosphorylation at 1 h in CD14 monocytes relative to baseline indicates a shorter period of time to recovery from pain or functional impairment.

In some specific embodiments, a decrease in pNF-κB levels in monocytes, e.g. CD14+ monocytes immediately following surgery, for example within about 15 minutes, within about 30 minutes, within about 45 minutes, within about 1 hour, within about 1.5 hours, within about 2 hours compared to a pre-surgery base line level, is indicative that an individual will require a shorter period of time to achieve recovery, as assessed, for example, by time to mild pain, relative to an individual that does not show a significant decrease in pNF-κB.

When necessary, cells are dispersed into a single cell suspension, e.g. by enzymatic digestion with a suitable protease, e.g. collagenase, dispase, etc; and the like. An appropriate solution is used for dispersion or suspension. Such solution will generally be a balanced salt solution, e.g. normal saline, PBS, Hanks balanced salt solution, etc., conveniently supplemented with fetal calf serum or other naturally occurring factors, in conjunction with an acceptable buffer at low concentration, generally from 5-25 mM. Convenient buffers include HEPES1 phosphate buffers, lactate buffers, etc. The cells can be fixed, e.g. with 3% paraformaldehyde, and are usually permeabilized, e.g. with ice cold methanol; HEPES-buffered PBS containing 0.1% saponin, 3% BSA; covering for 2 min in acetone at −200 C; and the like as known in the art and according to the methods described herein.

In some embodiments, one or more cells are contained in a well of a 96 well plate or other commercially available multiwell plate. In an alternate embodiment, the reaction mixture or cells are in a cytometric measurement device. Other multiwell plates useful in the present invention include, but are not limited to 384 well plates and 1536 well plates. Still other vessels for containing the reaction mixture or cells and useful in the present invention will be apparent to the skilled artisan.

In some embodiment, the methods of the invention include the use of liquid handling components. The liquid handling systems can include robotic systems comprising any number of components. In addition, any or all of the steps outlined herein can be automated; thus, for example, the systems can be completely or partially automated. See U.S. Ser. No. 61/048,657. As will be appreciated by those in the art, there are a wide variety of components which can be used, including, but not limited to, one or more robotic arms; plate handlers for the positioning of microplates; automated lid or cap handlers to remove and replace lids for wells on non-cross contamination plates; tip assemblies for sample distribution with disposable tips; washable tip assemblies for sample distribution; 96 well loading blocks; cooled reagent racks; microtiter plate pipette positions (optionally cooled); stacking towers for plates and tips; and computer systems.

Fully robotic or microfluidic systems include automated liquid-, particle-, cell- and organism-handling including high throughput pipetting to perform all steps of screening applications. This includes liquid, particle, cell, and organism manipulations such as aspiration, dispensing, mixing, diluting, washing, accurate volumetric transfers; retrieving, and discarding of pipet tips; and repetitive pipetting of identical volumes for multiple deliveries from a single sample aspiration. These manipulations are cross-contamination-free liquid, particle, cell, and organism transfers. This instrument performs automated replication of microplate samples to filters, membranes, and/or daughter plates, high-density transfers, full-plate serial dilutions, and high capacity operation.

In some embodiments, platforms for multi-well plates, multi-tubes, holders, cartridges, minitubes, deep-well plates, microfuge tubes, cryovials, square well plates, filters, chips, optic fibers, beads, and other solid-phase matrices or platform with various volumes are accommodated on an upgradable modular platform for additional capacity. This modular platform includes a variable speed orbital shaker, and multi-position work decks for source samples, sample and reagent dilution, assay plates, sample and reagent reservoirs, pipette tips, and an active wash station. In some embodiments, the methods of the invention include the use of a plate reader.

In some embodiments, interchangeable pipet heads (single or multi-channel) with single or multiple magnetic probes, affinity probes, or pipetters robotically manipulate the liquid, particles, cells, and organisms. Multi-well or multi-tube magnetic separators or platforms manipulate liquid, particles, cells, and organisms in single or multiple sample formats.

In some embodiments, the instrumentation will include a detector, which can be a wide variety of different detectors, depending on the labels and assay. In some embodiments, useful detectors include a microscope(s) with multiple channels of fluorescence; plate readers to provide fluorescent, ultraviolet and visible spectrophotometric detection with single and dual wavelength endpoint and kinetics capability, fluorescence resonance energy transfer (FRET), luminescence, quenching, two-photon excitation, and intensity redistribution; CCD cameras to capture and transform data and images into quantifiable formats; and a computer workstation.

In some embodiments, the robotic apparatus includes a central processing unit which communicates with a memory and a set of input/output devices (e.g., keyboard, mouse, monitor, printer, etc.) through a bus. Again, as outlined below, this can be in addition to or in place of the CPU for the multiplexing devices of the invention. The general interaction between a central processing unit, a memory, input/output devices, and a bus is known in the art. Thus, a variety of different procedures, depending on the experiments to be run, are stored in the CPU memory.

The differential presence of these markers is shown to provide for prognostic evaluations to detect individuals having clinical subtypes that correspond to longer or shorter recovery periods. In general, such prognostic methods involve determining the presence or level of activated signaling proteins in an individual sample of immune cells. Detection can utilize one or a panel of specific binding members, e.g. a panel or cocktail of binding members specific for one, two, three, four, five or more markers.

### Data Analysis

A signature pattern can be generated from a biological sample using any convenient protocol, for example as described below. The readout can be a mean, average, median or the variance or other statistically or mathematically-derived value associated with the measurement. The marker readout information can be further refined by direct comparison with the corresponding reference or control pattern. A binding pattern can be evaluated on a number of points: to determine if there is a statistically significant change at any point in the data matrix relative to a reference value; whether the change is an increase or decrease in the binding; whether the change is specific for one or more physiological states, and the like. The absolute values obtained for each marker under identical conditions will display a variability that is inherent in live biological systems and also reflects the variability inherent between individuals.

Following obtainment of the signature pattern from the sample being assayed, the signature pattern can be compared with a reference or base line profile to make a prognosis regarding the phenotype of the patient from which the sample was obtained/derived. Additionally, a reference or control signature pattern can be a signature pattern that is obtained from a sample of a patient known to correspond to longer or shorter recovery periods, and therefore can be a positive reference or control profile.

In certain embodiments, the obtained signature pattern is compared to a single reference/control profile to obtain information regarding the phenotype of the patient being assayed. In yet other embodiments, the obtained signature pattern is compared to two or more different reference/control profiles to obtain more in depth information regarding the phenotype of the patient. For example, the obtained signature pattern can be compared to a positive and negative reference profile to obtain confirmed information regarding whether the patient has the phenotype of interest.

Samples can be obtained from the tissues or fluids of an individual. For example, samples can be obtained from whole blood, tissue biopsy, serum, etc. Other sources of samples are body fluids such as lymph, cerebrospinal fluid, and the like. Also included in the term are derivatives and fractions of such cells and fluids

In order to identify profiles that are indicative of responsiveness, a statistical test can provide a confidence level for a change in the level of markers between the test and reference profiles to be considered significant. The raw data can be initially analyzed by measuring the values for each marker, usually in duplicate, triplicate, quadruplicate or in 5-10 replicate features per marker. A test dataset is considered to be different than a reference dataset if one or more of the parameter values of the profile exceeds the limits that correspond to a predefined level of significance.

To provide significance ordering, the false discovery rate (FDR) can be determined. First, a set of null distributions of dissimilarity values is generated. In one embodiment, the values of observed profiles are permuted to create a sequence of distributions of correlation coefficients obtained out of chance, thereby creating an appropriate set of null distributions of correlation coefficients (see Tusher et al. (2001) PNAS 98, 5116-21, herein incorporated by reference). This analysis algorithm is currently available as a software “plug-in” for Microsoft Excel know as Significance Analysis of Microarrays (SAM). The set of null distribution is obtained by: permuting the values of each profile for all available profiles; calculating the pair-wise correlation coefficients for all profile; calculating the probability density function of the correlation coefficients for this permutation; and repeating the procedure for N times, where N is a large number, usually 300. Using the N distributions, one calculates an appropriate measure (mean, median, etc.) of the count of correlation coefficient values that their values exceed the value (of similarity) that is obtained from the distribution of experimentally observed similarity values at given significance level.

The FDR is the ratio of the number of the expected falsely significant correlations (estimated from the correlations greater than this selected Pearson correlation in the set of randomized data) to the number of correlations greater than this selected Pearson correlation in the empirical data (significant correlations). This cut-off correlation value can be applied to the correlations between experimental profiles.

For SAM, Z-scores represent another measure of variance in a dataset, and are equal to a value of X minus the mean of X, divided by the standard deviation. A Z-Score tells how a single data point compares to the normal data distribution. A Z-score demonstrates not only whether a datapoint lies above or below average, but how unusual the measurement is. The standard deviation is the average distance between each value in the dataset and the mean of the values in the dataset.

Using the aforementioned distribution, a level of confidence is chosen for significance. This is used to determine the lowest value of the correlation coefficient that exceeds the result that would have obtained by chance. Using this method, one obtains thresholds for positive correlation, negative correlation or both. Using this threshold(s), the user can filter the observed values of the pairwise correlation coefficients and eliminate those that do not exceed the threshold(s). Furthermore, an estimate of the false positive rate can be obtained for a given threshold. For each of the individual “random correlation” distributions, one can find how many observations fall outside the threshold range. This procedure provides a sequence of counts. The mean and the standard deviation of the sequence provide the average number of potential false positives and its standard deviation. Alternatively, any convenient method of statistical validation can be used.

The data can be subjected to non-supervised hierarchical clustering to reveal relationships among profiles. For example, hierarchical clustering can be performed, where the Pearson correlation is employed as the clustering metric. One approach is to consider a patient disease dataset as a “learning sample” in a problem of “supervised learning”. CART is a standard in applications to medicine (Singer (1999) Recursive Partitioning in the Health Sciences, Springer), which can be modified by transforming any qualitative features to quantitative features; sorting them by attained significance levels, evaluated by sample reuse methods for Hotelling's T² statistic; and suitable application of the lasso method. Problems in prediction are turned into problems in regression without losing sight of prediction, indeed by making suitable use of the Gini criterion for classification in evaluating the quality of regressions.

Other methods of analysis that can be used include logistic regression. One method of logic regression Ruczinski (2003) Journal of Computational and Graphical Statistics 12:475-512. Logic regression resembles CART in that its classifier can be displayed as a binary tree. It is different in that each node has Boolean statements about features that are more general than the simple “and” statements produced by CART.

Another approach is that of nearest shrunken centroids (Tibshirani (2002) PNAS 99:6567-72). The technology is k-means-like, but has the advantage that by shrinking cluster centers, one automatically selects features (as in the lasso) so as to focus attention on small numbers of those that are informative. The approach is available as Prediction Analysis of Microarrays (PAM) software, a software “plug-in” for Microsoft Excel, and is widely used. Two further sets of algorithms are random forests (Breiman (2001) Machine Learning 45:5-32 and MART (Hastie (2001) The Elements of Statistical Learning, Springer). These two methods are already “committee methods.” Thus, they involve predictors that “vote” on outcome. Several of these methods are based on the “R” software, developed at Stanford University, which provides a statistical framework that is continuously being improved and updated in an ongoing basis.

Other statistical analysis approaches including principle components analysis, recursive partitioning, predictive algorithms, Bayesian networks, and neural networks.

These tools and methods can be applied to several classification problems. For example, methods can be developed from the following comparisons: i) all cases versus all controls, ii) all cases versus nonresponsive controls, iii) all cases versus responsive controls.

In a second analytical approach, variables chosen in the cross-sectional analysis are separately employed as predictors. Given the specific outcome, the random lengths of time each patient will be observed, and selection of proteomic and other features, a parametric approach to analyzing responsiveness can be better than the widely applied semi-parametric Cox model. A Weibull parametric fit of survival permits the hazard rate to be monotonically increasing, decreasing, or constant, and also has a proportional hazards representation (as does the Cox model) and an accelerated failure-time representation. All the standard tools available in obtaining approximate maximum likelihood estimators of regression coefficients and functions of them are available with this model.

In addition the Cox models can be used, especially since reductions of numbers of covariates to manageable size with the lasso will significantly simplify the analysis, allowing the possibility of an entirely nonparametric approach to survival.

These statistical tools are applicable to all manner of marker expression data. A set of data that can be easily determined, and that is highly informative regarding detection of individuals with clinically significant time of recovery from surgery is provided.

Also provided are databases of signature patterns for prognosis for time of recovery. Such databases will typically comprise signature patterns of individuals having shorter and longer times to recovery, etc., where such profiles are as described above.

The analysis and database storage can be implemented in hardware or software, or a combination of both. In one embodiment of the invention, a machine-readable storage medium is provided, the medium comprising a data storage material encoded with machine readable data which, when using a machine programmed with instructions for using said data, is capable of displaying a any of the datasets and data comparisons of this invention. Such data can be used for a variety of purposes, such as patient monitoring, initial diagnosis, and the like. Preferably, the invention is implemented in computer programs executing on programmable computers, comprising a processor, a data storage system (including volatile and non-volatile memory and/or storage elements), at least one input device, and at least one output device. Program code is applied to input data to perform the functions described above and generate output information. The output information is applied to one or more output devices, in known fashion. The computer can be, for example, a personal computer, microcomputer, or workstation of conventional design.

Each program is preferably implemented in a high level procedural or object oriented programming language to communicate with a computer system. However, the programs can be implemented in assembly or machine language, if desired. In any case, the language can be a compiled or interpreted language. Each such computer program is preferably stored on a storage media or device (e.g., ROM or magnetic diskette) readable by a general or special purpose programmable computer, for configuring and operating the computer when the storage media or device is read by the computer to perform the procedures described herein. The system can also be considered to be implemented as a computer-readable storage medium, configured with a computer program, where the storage medium so configured causes a computer to operate in a specific and predefined manner to perform the functions described herein.

A variety of structural formats for the input and output means can be used to input and output the information in the computer-based systems of the present invention. One format for an output means test datasets possessing varying degrees of similarity to a trusted profile. Such presentation provides a skilled artisan with a ranking of similarities and identifies the degree of similarity contained in the test pattern.

The signature patterns and databases thereof can be provided in a variety of media to facilitate their use. “Media” refers to a manufacture that contains the signature pattern information of the present invention. The databases of the present invention can be recorded on computer readable media, e.g. any medium that can be read and accessed directly by a computer. Such media include, but are not limited to: magnetic storage media, such as floppy discs, hard disc storage medium, and magnetic tape; optical storage media such as CD-ROM; electrical storage media such as RAM and ROM; and hybrids of these categories such as magnetic/optical storage media. One of skill in the art can readily appreciate how any of the presently known computer readable mediums can be used to create a manufacture comprising a recording of the present database information. “Recorded” refers to a process for storing information on computer readable medium, using any such methods as known in the art. Any convenient data storage structure can be chosen, based on the means used to access the stored information. A variety of data processor programs and formats can be used for storage, e.g. word processing text file, database format, etc.

### Kits

In some embodiments, the invention provides kits for the classification, diagnosis, prognosis, theranosis, and/or prediction of an outcome following surgery in a subject. The kit may further comprise a software package for data analysis of the cellular state and its physiological status, which may include reference profiles for comparison with the test profile and comparisons to other analyses as referred to above. The kit may also include instructions for use for any of the above applications.

Kits provided by the invention may comprise one or more of the affinity reagents described herein, such as phospho-specific antibodies and antibodies that distinguish subsets of immune cells. A kit may also include other reagents that are useful in the invention, such as modulators, fixatives, containers, plates, buffers, therapeutic agents, instructions, and the like.

Kits provided by the invention can comprise one or more labeling elements. Non-limiting examples of labeling elements include small molecule fluorophores, proteinaceous fluorophores, radioisotopes, enzymes, antibodies, chemiluminescent molecules, biotin, streptavidin, digoxigenin, chromogenic dyes, luminescent dyes, phosphorous dyes, luciferase, magnetic particles, beta-galactosidase, amino groups, carboxy groups, maleimide groups, oxo groups and thiol groups, quantum dots, chelated or caged lanthanides, isotope tags, radiodense tags, electron-dense tags, radioactive isotopes, paramagnetic particles, agarose particles, mass tags, e-tags, nanoparticles, and vesicle tags.

In some embodiments, the kits of the invention enable the detection of signaling proteins by sensitive cellular assay methods, such as IHC and flow cytometry, which are suitable for the clinical detection, classification, diagnosis, prognosis, theranosis, and outcome prediction.

Such kits may additionally comprise one or more therapeutic agents. The kit may further comprise a software package for data analysis of the physiological status, which may include reference profiles for comparison with the test profile.

Such kits may also include information, such as scientific literature references, package insert materials, clinical trial results, and/or summaries of these and the like, which indicate or establish the activities and/or advantages of the composition, and/or which describe dosing, administration, side effects, drug interactions, or other information useful to the health care provider. Such information may be based on the results of various studies, for example, studies using experimental animals involving in vivo models and studies based on human clinical trials. Kits described herein can be provided, marketed and/or promoted to health providers, including physicians, nurses, pharmacists, formulary officials, and the like. Kits may also, in some embodiments, be marketed directly to the consumer.

### Reports

In some embodiments, providing an evaluation of a subject for a classification, diagnosis, prognosis, theranosis, and/or prediction of an outcome following surgery includes generating a written report that includes the artisan's assessment of the subject's state of health i.e. a “diagnosis assessment”, of the subject's prognosis, i.e. a “prognosis assessment”, and/or of possible treatment regimens, i.e. a “treatment assessment”. Thus, a subject method may further include a step of generating or outputting a report providing the results of a diagnosis assessment, a prognosis assessment, or treatment assessment, which report can be provided in the form of an electronic medium (e.g., an electronic display on a computer monitor), or in the form of a tangible medium (e.g., a report printed on paper or other tangible medium).

A “report,” as described herein, is an electronic or tangible document which includes report elements that provide information of interest relating to a diagnosis assessment, a prognosis assessment, and/or a treatment assessment and its results. A subject report can be completely or partially electronically generated. A subject report includes at least a diagnosis assessment, i.e. a diagnosis as to whether a subject will have a particular clinical response to surgical treatment, and/or a suggested course of treatment to be followed. For example, a decision can be made as to whether the subject will benefit from surgical intervention. A subject report can further include one or more of: 1) information regarding the testing facility; 2) service provider information; 3) subject data; 4) sample data; 5) an assessment report, which can include various information including: a) test data, where test data can include an analysis of cellular signaling responses to activation, b) reference values employed, if any.

The report may include information about the testing facility, which information is relevant to the hospital, clinic, or laboratory in which sample gathering and/or data generation was conducted. This information can include one or more details relating to, for example, the name and location of the testing facility, the identity of the lab technician who conducted the assay and/or who entered the input data, the date and time the assay was conducted and/or analyzed, the location where the sample and/or result data is stored, the lot number of the reagents (e.g., kit, etc.) used in the assay, and the like. Report fields with this information can generally be populated using information provided by the user.

The report may include information about the service provider, which may be located outside the healthcare facility at which the user is located, or within the healthcare facility. Examples of such information can include the name and location of the service provider, the name of the reviewer, and where necessary or desired the name of the individual who conducted sample gathering and/or data generation. Report fields with this information can generally be populated using data entered by the user, which can be selected from among pre-scripted selections (e.g., using a drop-down menu). Other service provider information in the report can include contact information for technical information about the result and/or about the interpretive report.

The report may include a subject data section, including subject medical history as well as administrative subject data (that is, data that are not essential to the diagnosis, prognosis, or treatment assessment) such as information to identify the subject (e.g., name, subject date of birth (DOB), gender, mailing and/or residence address, medical record number (MRN), room and/or bed number in a healthcare facility), insurance information, and the like), the name of the subject's physician or other health professional who ordered the susceptibility prediction and, if different from the ordering physician, the name of a staff physician who is responsible for the subject's care (e.g., primary care physician).

The report may include a sample data section, which may provide information about the biological sample analyzed, such as the source of biological sample obtained from the subject (e.g. blood, type of tissue, etc.), how the sample was handled (e.g. storage temperature, preparatory protocols) and the date and time collected. Report fields with this information can generally be populated using data entered by the user, some of which may be provided as pre-scripted selections (e.g., using a drop-down menu).

The report may include an assessment report section, which may include information generated after processing of the data as described herein. The interpretive report can include a prognosis of the likelihood that the patient will have a surgery-attributable death or progression. The interpretive report can include, for example, results of the analysis, methods used to calculate the analysis, and interpretation, i.e. prognosis. The assessment portion of the report can optionally also include a Recommendation(s). For example, where the results indicate the subject's prognosis for time to recovery.

It will also be readily appreciated that the reports can include additional elements or modified elements. For example, where electronic, the report can contain hyperlinks which point to internal or external databases which provide more detailed information about selected elements of the report. For example, the patient data element of the report can include a hyperlink to an electronic patient record, or a site for accessing such a patient record, which patient record is maintained in a confidential database. This latter embodiment may be of interest in an in-hospital system or in-clinic setting. When in electronic format, the report is recorded on a suitable physical medium, such as a computer readable medium, e.g., in a computer memory, zip drive, CD, DVD, etc.

It will be readily appreciated that the report can include all or some of the elements above, with the proviso that the report generally includes at least the elements sufficient to provide the analysis requested by the user (e.g., a diagnosis, a prognosis, or a prediction of responsiveness to a therapy).

### EXPERIMENTAL

The following examples are given for the purpose of illustrating various embodiments of the invention and are not meant to limit the present invention in any fashion. The present examples, along with the methods described herein are presently representative of preferred embodiments, are exemplary, and are not intended as limitations on the scope of the invention. Changes therein and other uses which are encompassed within the spirit of the invention as defined by the scope of the claims will occur to those skilled in the art.

### Example 1

### Single-Cell Deep Immune Profiling by Mass Cytometry Reveals Trauma-Specific Immune Signatures that Contain Surgical Recovery Correlates

Delayed recovery from surgery causes substantial personal suffering, with consequent societal and economic costs. The extent to which immune mechanisms determine recovery after surgical trauma remain ill-defined. Single-cell mass cytometry was utilized to measure the expression levels of 35 cell-surface proteins and intracellular phospho-specific epitopes in serial whole blood samples collected from 32 patients undergoing primary hip replacement. The simultaneous analysis of 14,000 phosphorylation events across 8 immune cell subsets revealed remarkably uniform signaling responses among patients, demarcating a “trauma-specific” immune signature.

When regressed against clinical parameters of surgical recovery, including functional impairment and pain, strong positive correlations were found with STAT3, CREB and NF-κB signaling responses in subsets of CD14+ monocytes (R=0.7-0.8, False Discovery Rate <0.01). These mechanistically derived immune correlates hold promise for guiding diagnostic and therapeutic strategies that may improve postoperative recovery in surgical patients.

More than 100 million surgeries are performed annually in Europe and the United States. This number is expected to grow as the population ages. Convalescence after surgery is highly variable, and delays in recovery result in personal suffering and societal and economic costs. Perioperative care now includes enhanced-recovery protocols and evidence-based practice guidelines largely anchored in observational data. The physiologic and mechanistic underpinnings of surgical recovery remain a “black box” phenomenon, however. Understanding the mechanisms that drive recovery after surgery will advance therapeutic strategies and allow patient-specific tailoring of recovery protocols. Tissue injury mediates a profound inflammatory response, that has prompted a long-standing interest in understanding how the immune system determines recovery from surgical trauma. Previous studies examining the immune response to surgery or major trauma, focused on secreted humoral factors, distribution patterns of immune cell subsets_{9,10}, and genomic analysis of pooled circulating leukocytes. These reports provided important insight into mechanisms governing the inflammatory response to traumatic injury but did not reveal strong correlates with clinical recovery. Although weak correlates to clinical outcomes were reported, none of these studies measured immune responses at the single-cell level, and stronger signals might have gone undetected as specific immune cell subsets would have been phenotypically under-characterized.

Traumatic injury initiates an intricate programmed immune response: Hours following severe trauma, neutrophils and monocytes are rapidly activated and recruited to the periphery by damage response antigens, alarmins (e.g., HMGB1), and increased levels of TNFα, IL-1β, IL-6. This is followed by a compensatory phase characterized by decreased numbers of T cell subsets. The various immune cell types are thought to integrate multiple environmental signals into cohesive signaling responses that enable wound healing and recovery. A recent genome-wide analysis of pooled circulating leukocytes revealed that traumatic injury organized more than 80% of the leukocyte transcriptome according to cell type-specific signaling pathways. A retrospective analysis of these data identified an expression pattern in a subset of genes that differentiated extremes of clinical recovery.

Here mass cytometry, a highly parameterized single-cell based platform that can determine functional responses in precisely phenotyped immune cell subsets, was employed to identify cell subsets and corresponding signaling pathways that correlate with clinical recovery. The expression levels of 35 cell-surface proteins and intracellular phospho-specific epitopes were simultaneously measured at 1 h, 24 h, 72 h, and 6 weeks after surgery in whole blood samples from 32 patients undergoing primary hip arthroplasty. During a 6-week post-surgery observation period, functional recovery and pain, the major determinants of clinical recovery, were evaluated. Highly regimented changes in the distribution of immune cells were observed in conjunction with cell-type specific signaling responses that demarcated a “trauma”-specific immune signature. When regressed against parameters of clinical recovery, strong correlates were found within signaling responses of specific cell subsets rather than in frequency changes of immune cell subsets. While the profiling was accomplished with 35 mass cytometry markers, it is important to note that the principle component “diagnostic” can be reduced to as few as 5-7 markers on conventional fluorescence based clinical flow instruments. Notably, all signaling responses correlating with clinical recovery occurred in subsets of CD14+ monocytes, underscoring a central role of these cells in processes enabling or disabling recovery from surgery.

### Results

Mass Cytometry Assay Performance in Clinical Samples.

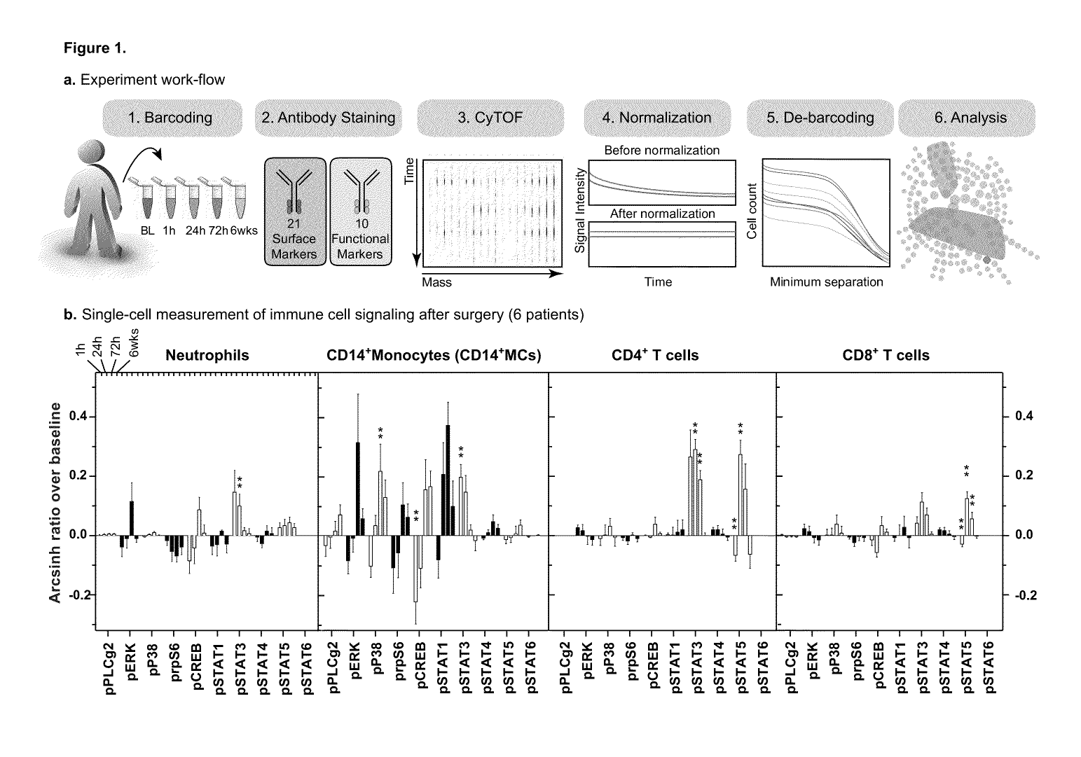

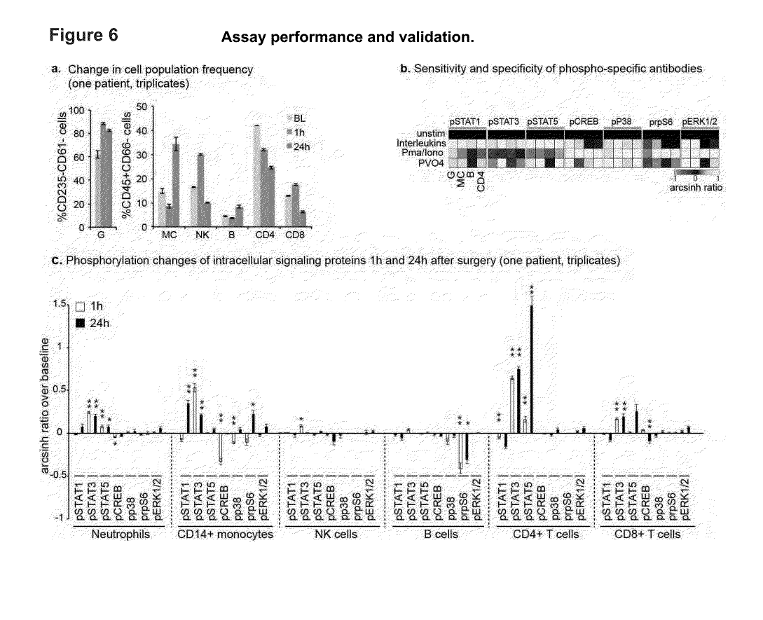

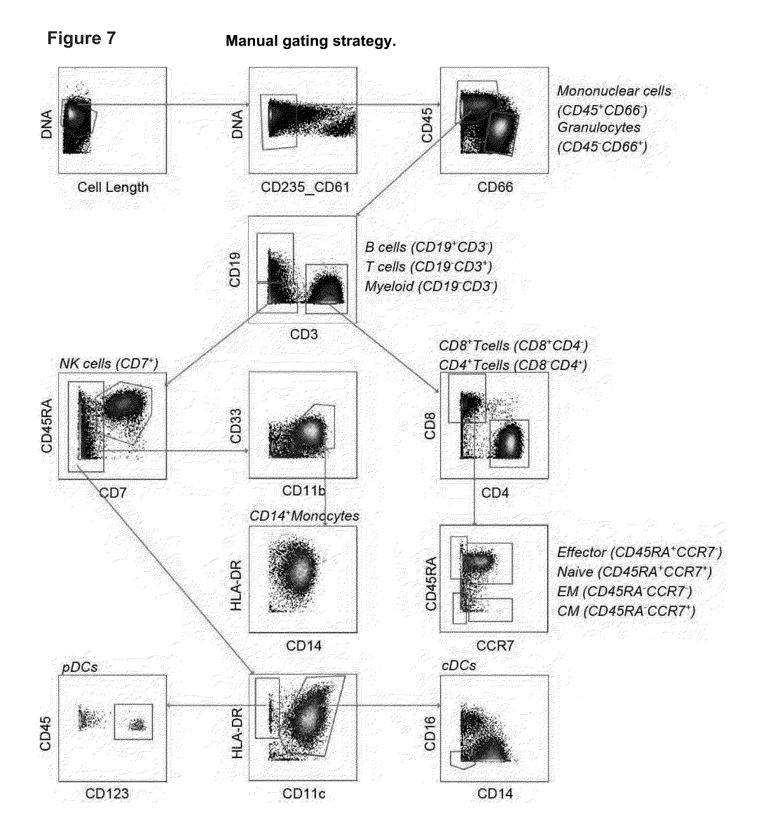

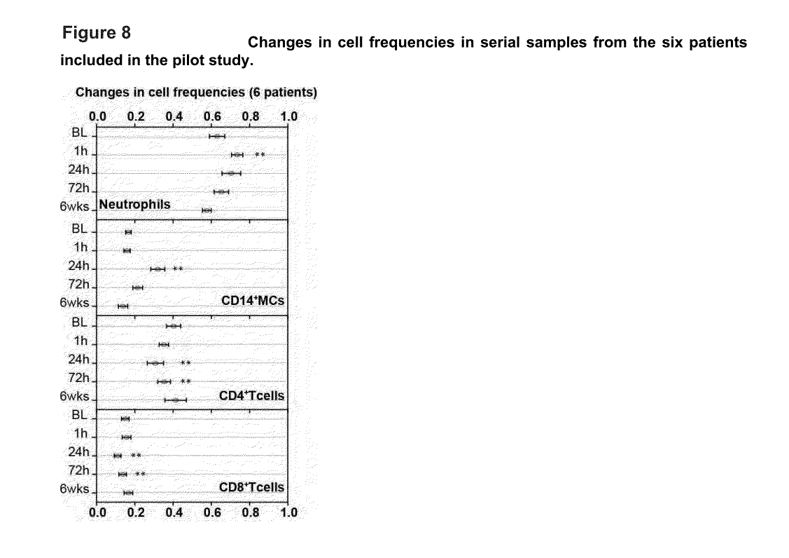

Based on a premise that surgical intervention, or “trauma”, acts as a systemic perturbation on multiple physiologic processes in the body, cell subsets based on traditional surface marker phenotyping were analyzed simultaneously with intracellular signaling cascades downstream of activated receptors. Whole peripheral blood was collected from primary hip arthroplasty (PHA) patients and, critically, was processed within 30 minutes to remain as close as possible to in vivo conditions. In a preliminary phase, samples from one patient collected 1 h before and 1 and 24 h after surgery were assayed in triplicate to determine whether trauma-induced changes in immune cell frequencies and intracellular signaling responses (phosphorylation of signaling proteins) could reliably be detected with mass cytometry. Reproducible changes were observed for cell frequencies and intracellular signaling responses, validating the assay for subsequent studies (FIG. 6). In a pilot study of six PHA patients, whole blood samples were collected 1 h before and 1 h, 24 h, 72 h, and 6 weeks after surgery. Samples were barcoded using a combination of stable isotope mass-tags20, stained with antibodies recognizing 21 cell-surface proteins and phospho-epitopes of 10 intracellular proteins, and processed for mass cytometry (FIG. 1a, Table 2). Analysis initially focused on neutrophils, CD14⁺ monocytes (CD14⁺MCs), and CD4⁺ and CD8⁺ T cells (FIG. 7). Consistent with previous reports, surgery induced a 1.2-fold (±0.06, q<0.01) expansion of neutrophils 1 h after surgery, a 1.9-fold (±0.19, q<0.01) expansion of CD14⁺MCs at 24 h, and a contraction of CD4⁺ and CD8⁺ T cells to 0.77-fold (±0.07, q<0.01) and 0.71-fold (±0.07, q<0.01), respectively, at 24 h (FIG. 8). Intracellular signaling responses, indicated by changes in phosphorylation of STAT1, STAT3, STATS, CREB, and p38, were induced in time and cell-type specific manners (FIG. 1b, q<0.01). Six weeks after surgery, cell-subset frequencies and magnitudes of phospho-signals did not differ from pre-surgical values (q>0.05), indicating restoration of immune homeostasis.

CD33⁺CD11b⁺CD14⁺HLA-DR^{low} Monocytes Expand 6-Fold after Surgery.

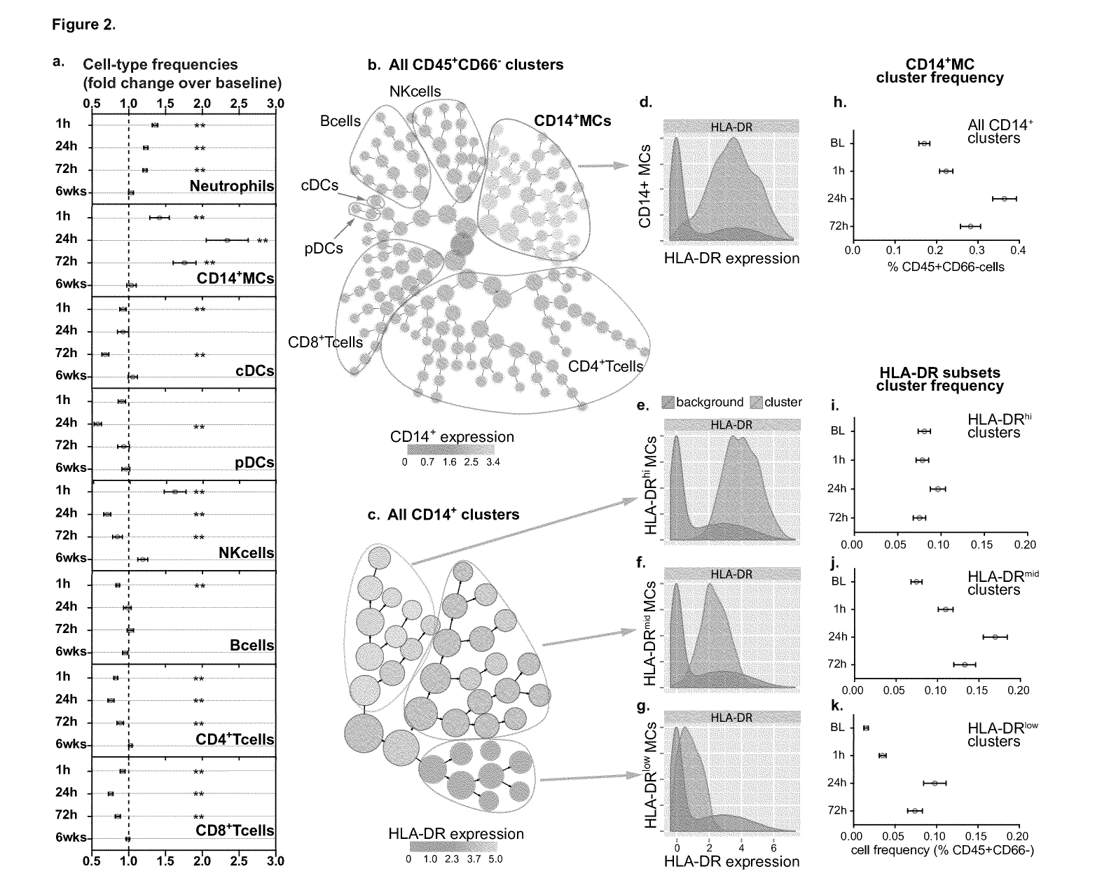

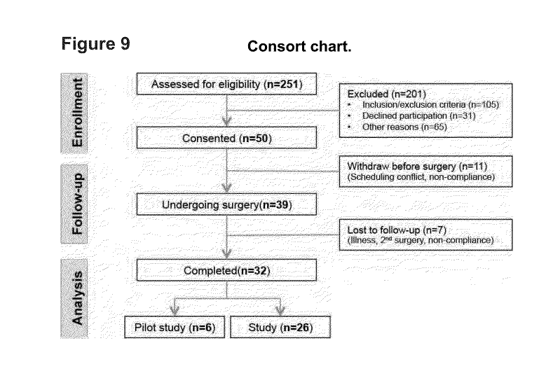

Having established that mass cytometry enabled the detection of surgery-induced perturbations in cell frequency and signaling, an observational study was conducted in twenty-six patients undergoing PHA. Serial blood samples and longitudinal data on clinical recovery were captured over a six-week period (Table 1, FIG. 9). Based on the pilot results, the antibody panel was modified to study specific signaling pathways in more detail and exclude noninformative antibodies (Table 2). The frequencies of neutrophils, CD14⁺MCs, classical dendritic cells (cDCs), plasmacytoid dendritic cells (pDCs), natural killer cells (NK), B cells, and CD4⁺ and CD8⁺ T cells were determined by manual gating (FIG. 2a, FIG. 7). Consistent with results from previous reports and the pilot study, NK cells (1.6-fold (±0.15, q<0.01)) and neutrophils (1.3-fold (±0.04, q<0.01)) expanded 1 h after surgery. CD14⁺MCs expanded 2.4-fold (±0.29, q<0.01) and 1.8-fold (±0.16 q<0.01) at 24 h and 72 h, respectively. Mobilization of the myeloid compartment was followed by a contraction at 24 h of CD4⁺ and CD8⁺ T cells to 0.76-fold (±0.04, q<0.01) and 0.72-fold (±0.03, q<0.01), respectively, that became less pronounced at 72 h (0.88±0.04 and 0.85±0.03, respectively, q<0.01).

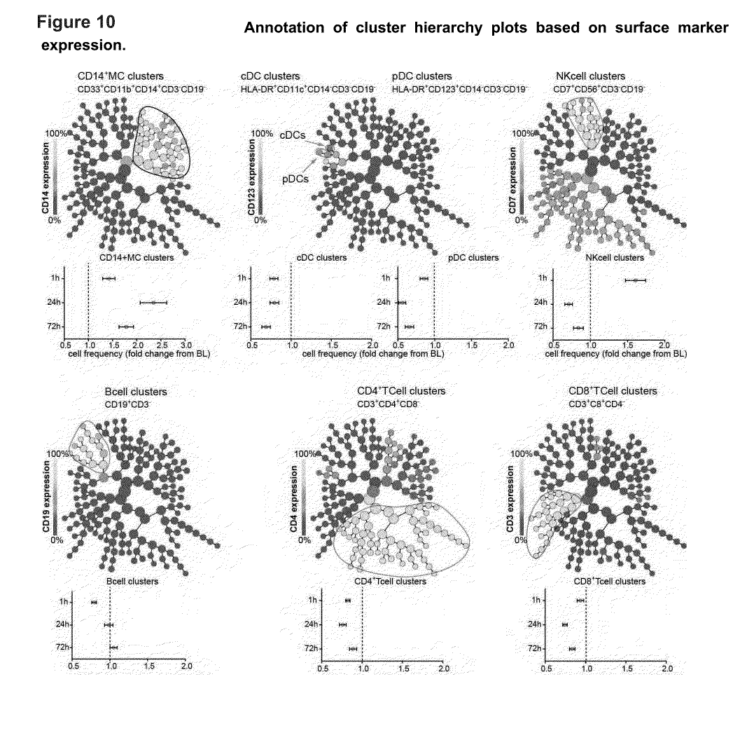

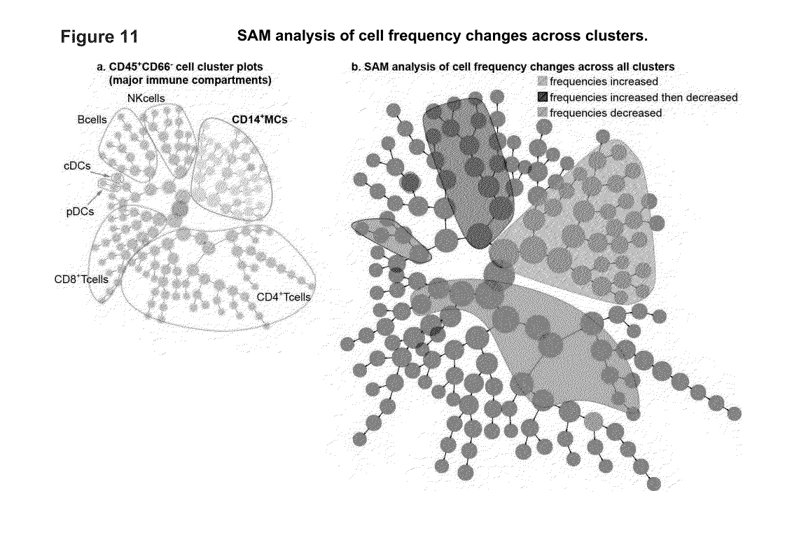

Consistent with pilot results, cell-type frequencies six weeks after surgery were similar to pre-surgical values (q>0.05). A major advantage afforded by high-dimensional multiparameter data analyses lies in the ability to detect finely tuned cell subsets with signaling changes that would be undetected in low parameter space. An unsupervised clustering algorithm was applied to comprehensively explore surgery-induced changes in cell subsets that may have been overlooked by manual gating strategies. The algorithm distills multidimensional single-cell data to a hierarchy of related clusters on the basis of cell surface markers (FIG. 2b, FIG. 10). Cluster-specific analysis of cell frequency changes revealed that clusters within the CD45⁺CD66⁻CD3⁻CD19⁻CD33⁺CD11b⁺CD14⁺ compartment (CD14⁺MC clusters) expanded 4.0-fold (±0.28) after surgery, more than any other cell cluster (FIG. 11).

Expression of the HLA-DR antigen partitioned CD14⁺MC clusters into HLADR^{hi}, HLA-DR^{mid}, and HLA-DR^{low} compartments (FIG. 2c-g). Quantification of CD14⁺MC cluster frequencies showed that the HLA-DR″^{d} and HLA-DR^{low} compartments accounted for 49% and 45% of the CD14⁺MC cluster expansion (FIG. 2h-k). Notably, CD33⁺CD11b⁺CD14⁺HLA-DR^{low} monocytes expanded 6-fold after surgery and had phenotypic similarity to myeloid derived suppressor cells (MDSC), previously described in the context of human malignancies as inhibitors of the adaptive immune system. Results of this unsupervised, highly parameterized analysis expand previous reports on monocytic HLA-DR expression after surgery. The current results underscore an unequivocal role of HLA-DR^{mid} and HLA-DR^{low} CD14⁺MCs in the healing process as they enable quantitative comparison among cell subsets within the broader context of the immune system.

STAT3, CREB and NF-κB Signaling Pathways are Differentially Activated in CD14+ Monocytes in Response to Surgery.

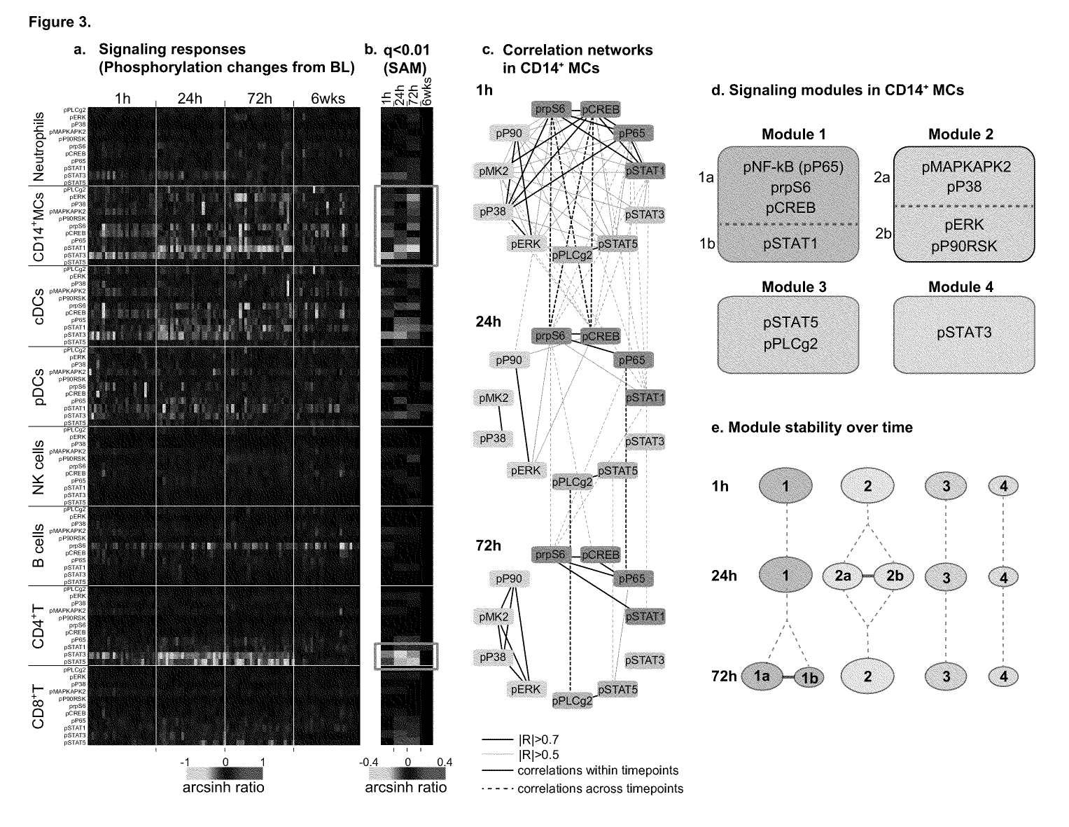

A visual synopsis of surgery-induced changes in the phosphorylation states of eleven intracellular signaling proteins across eight different immune cell subsets, at four time points, and in 26 patients is shown (FIG. 3a). Significance Analysis of Microarray (SAM) revealed 135 significant immune signaling responses to surgery (q<0.01) with cell-type specific distributions across major hematopoietic lineages (FIG. 3b). Notably, 97% of all phospho-signals detected 1 h before and 6 weeks after surgery were of similar magnitude (q>0.05), underscoring the tight regulation of the immune system and its ability to restore homeostasis (FIG. 3b).

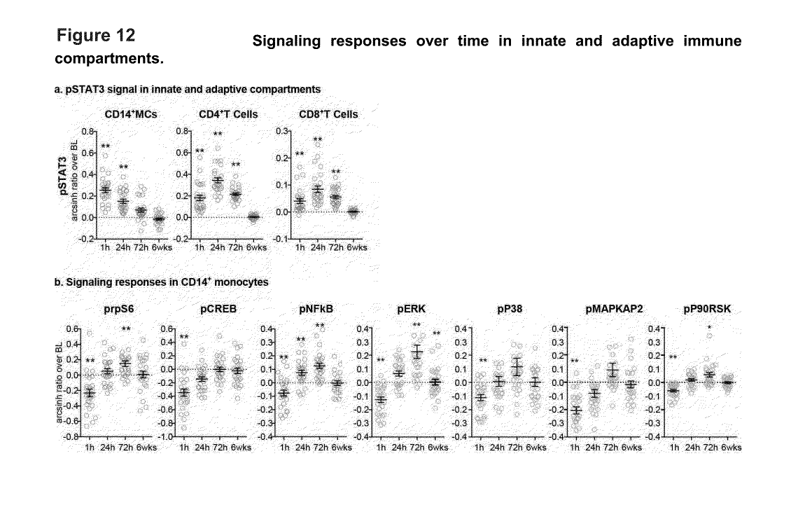

Between 1 h and 72 h after surgery, time-dependent signaling responses were detected in all immune cell types (Table 3). Signaling changes were most pronounced in CD14⁺MCs and CD4⁺ T cells (FIG. 3b, FIG. 12). Sequential activation of STAT3 and STAT1 characterized the STAT response in CD14⁺MCs, whereas activation of STAT3 and STATS characterized the STAT response in CD4⁺ T cells. Activation of STAT3 and STATS in CD4+ T cells was detected at 1 h; the highest level of activity of STAT3 in CD14⁺MCs was observed at this time point. Activity of STAT3 and STATS was less pronounced in CD8⁺ than CD4⁺ T cells but followed a similar pattern.

Results indicate early and concurrent activation of major signaling pathways in innate and adaptive immune cell compartments. This challenges the conventional view that innate and adaptive immune responses to surgical trauma follow a sequential temporal pattern. Further investigation of signaling responses in CD14⁺MCs revealed significant dephosphorylation of ERK, p38, MAPKAPK2, p90RSK, rpS6, CREB, and NF-κB (p65-RelA) at 1 h after surgery (FIG. 3a, 3b and FIG. 12). By 72 h, phosphorylation of these proteins had either returned to or exceeded baseline values.

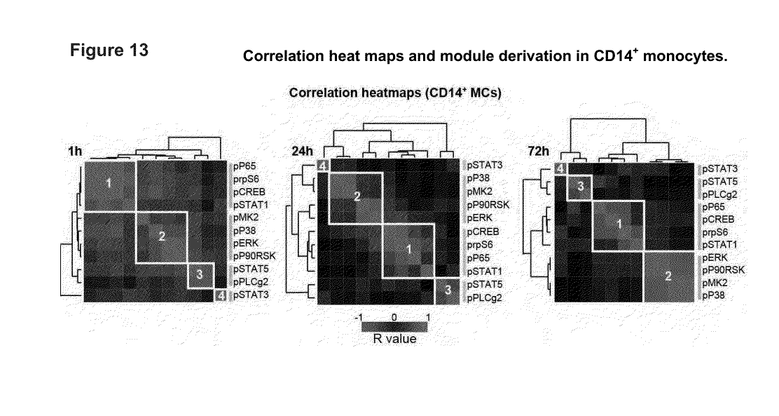

To characterize the signaling network underlying these coordinated phosphorylation patterns, correlation analysis was performed (FIG. 3c). Clustering of the resultant correlation coefficients revealed four modules that were preserved in all patients at all time points after surgery (FIG. 3d, 3e and FIG. 13). Module 1 consisted of pNF-κB, pCREB, and prpS6, and module 2 consisted of pp38, pMAPKAPK2, pERK, and pp90RSK. Each of these proteins can be activated downstream of Toll-like receptors known to play an essential role in the innate response to sterile inflammation. Module 1 also included STAT1, possibly reflecting the indirect regulation of STAT1 downstream of TLR4. Module 3 consisted of pSTAT5 and pPLCγ2, suggestive of coordinated activations of parallel signaling pathways not previously shown to cross-communicate. Module 4 consisted only of pSTAT3 and was anti-correlated with other modules; the pSTAT3 response may be linked to the known increase in plasma IL-6 concentration after surgery.

Signaling Responses in CD14+ Monocyte Subsets Correlate with Surgical Recovery.

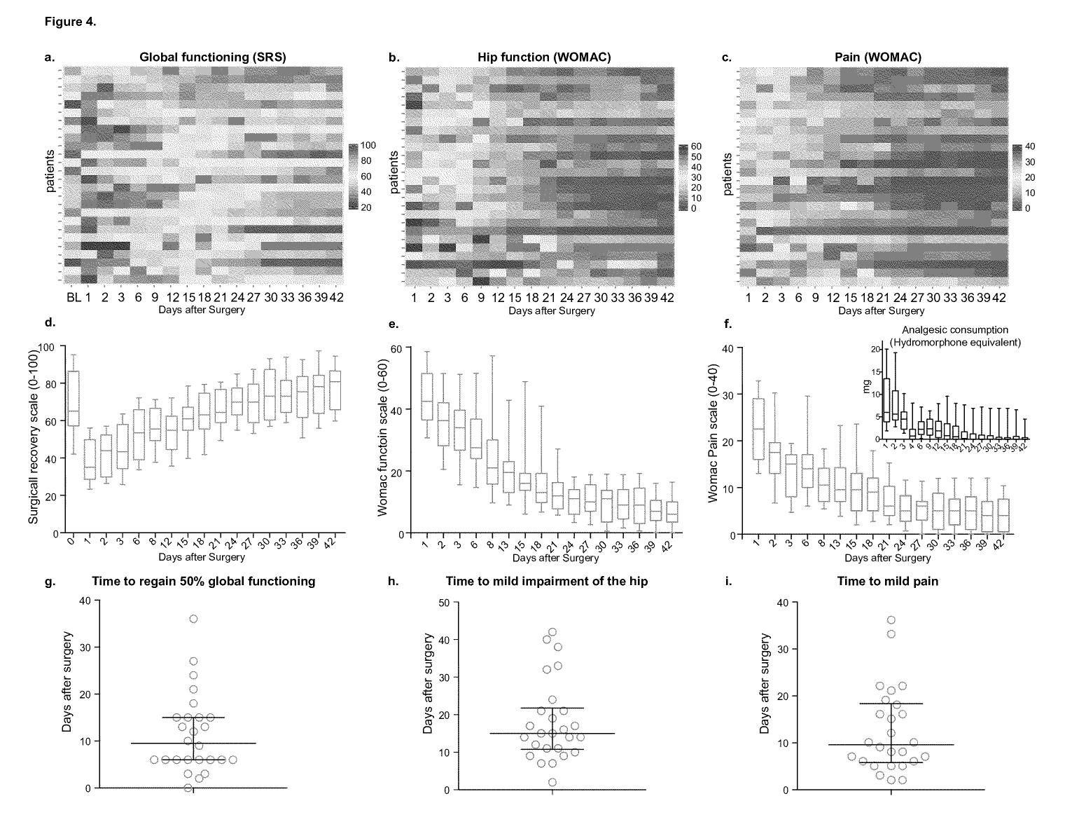

Considering that the inflammatory response to surgical trauma can engage as many as 40 receptors, the consistent integration of multiple environmental signals into cell type-specific signaling networks highlights the ability of the immune system to mount a remarkably uniform and “trauma”-specific response. The magnitude of this response varied among patients, which begs the question as to whether the variability between patients constitutes “noise” or, alternatively, reflects patient-specific differences that could correlate with differences in clinical recovery. Impaired functioning and pain after surgery critically determine when patients can resume normal activities. Heat maps depicting parameters of clinical recovery for individual patients during the six-week period following surgery reflect large inter-patient variability for three outcomes: global functioning, hip function, and pain (FIG. 4a-c). The median time to recuperate from impaired global functioning was 3 weeks. Clinical resolution of significant functional impairment of the hip (score ≦18) and pain (score ≦12) occurred during the second week (FIG. 4d-f).

However, the rate of recovery varied greatly among patients. The median time to regain 50% of global functioning was 10 days with a range of 0 to 36 days. The median time to experience only mild functional impairment of the hip was 15 days with a range of 2 to 42 days. The median time to suffer from only mild pain was 10 days with a similar wide range of 2 to 36 days (FIG. 4g-i). Among all demographic and clinical variables only gender was significantly related to a clinical recovery parameter. The median time to regain 50% of global functioning was 6 days (range 5-12) in women and 15 days (range 6-20) in men (p=0.02). Recovery of global functioning was not correlated with times to mild functional impairment of the hip or pain, but a significant correlation was detected between the times to mild functional impairment of the hip and pain (R=0.6, p=0.004). Thus, in this homogeneous patient population rates of recovery differed greatly.

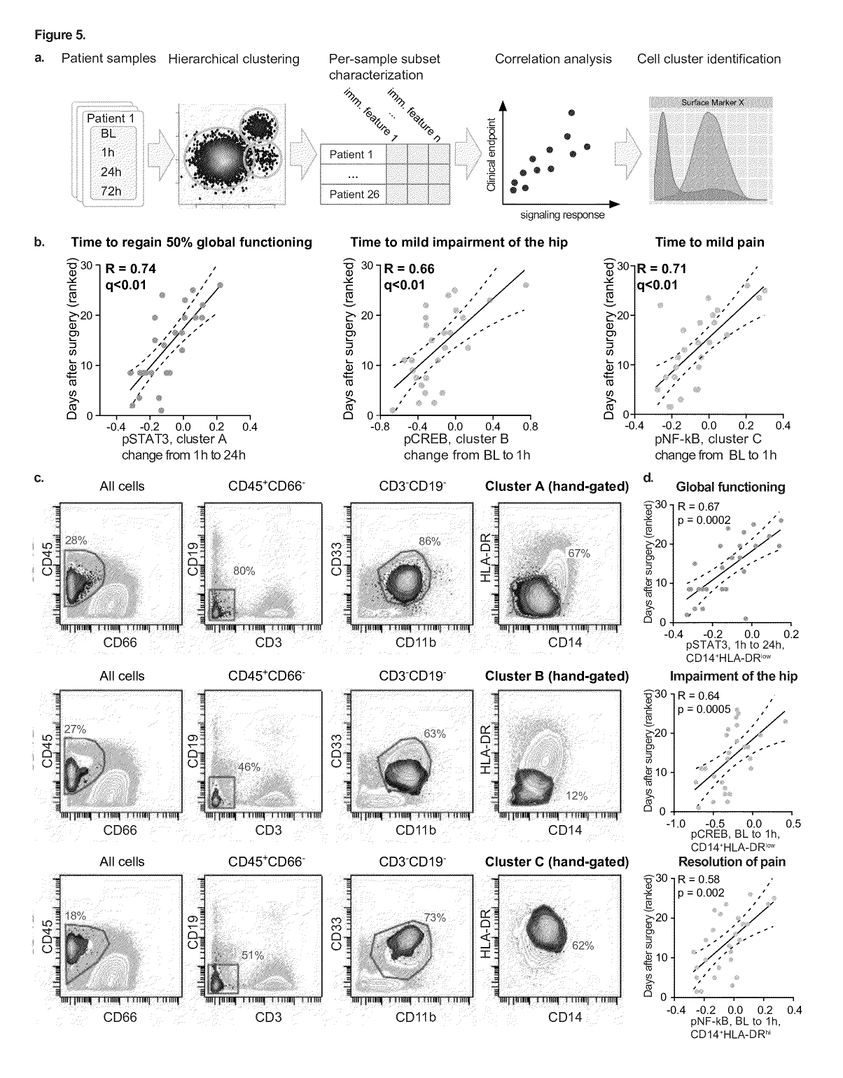

To gain an objective view of the relationships between the multidimensional mass cytometry dataset and clinical outcomes, a method for unsupervised identification of cellular responses associated with a clinical outcome was used (FIG. 5a). This algorithm demarks cell subsets using the hierarchical clustering described above (FIG. 2), attributes immune features (cell frequencies and signaling responses) to each cell cluster, and identifies significant associations (q<0.01) between immune features and parameters of clinical recovery using SAM. Significant correlations were detected for six immune cell features at a false discovery rate of 1% (R=0.66-0.80, Table 4). All were signaling responses in CD14⁺MC subsets (FIG. 5b, 5c). For instance, changes in STAT3 signaling between 1 h and 24 h in CD14⁺HLA-DR^{low/mid} MC clusters strongly correlated (R=0.72-0.80) with the time to regain 50% of global functioning (FIG. 5b, panel 1, FIG. 13). Changes in CREB signaling between baseline and 1 h in the CD14⁺HLA-DR^{low} MC cluster strongly correlated (R=0.66) with time to mild functional impairment of the hip (FIG. 5b, panel 2). Changes in NF-κB signaling between baseline and 1 h in the CD14⁺HLA-DR^{hi} MC cluster strongly correlated (R=0.71) with time to mild pain (FIG. 5b, panel 3). These correlations remained significant after correcting for demographic differences among study participants (Table 5) and were confirmed by manual gating (FIG. 5c, 5d). Thus, specific signaling responses in monocyte compartments are hallmarks of critical phenotypes defining clinical recovery and account for 40-60% of observed inter-patient variability.

Surgical trauma triggers a profound inflammatory response. The results provided herein demonstrate that specific immune response patterns underlie delayed recovery. In this patient cohort undergoing PHA, distinct signaling responses in CD14⁺ monocytes were identified that uniquely correlated with functional recovery and resolution of pain after surgery and accounted for 40-60% of observed patient variability. Mass cytometry provided high-dimensional numerical and functional characterization of the immune response to surgical trauma and enabled the detection of biological mechanisms critically associated with a health-relevant outcome. Using an unsupervised algorithmic approach, changes in cell frequencies and immune signaling responses at the single cell level were systematically evaluated across the entire immune system to identify immune correlates of clinical recovery.

Two themes evolved: 1) strong correlations were identified with signaling responses but not with changes in cell frequency and 2) signaling responses that correlated most significantly with clinical recovery occurred in CD14⁺ monocytes. The simultaneous monitoring of all major immune cell types provided a global view of surgery induced alterations across the immune system that included precisely timed changes in immune cell distribution and mobilization of distinct signaling networks in innate and adaptive compartments. Consistent with previous studies (see for example Rosenberger et al. (2009) The Journal of bone and joint surgery. American volume 91, 2783-2794; Hansbrough et al. (1984) American journal of surgery 148, 303-307; Slade et al. (1975) Surgery 78, 363-372; Ho et al. (2001) Blood 98, 140-145; Bocsi et al. (2011) Cytometry. Part B, Clinical cytometry 80, 212-220; Delogu et al. (2000) Arch Surg 135, 1141-1147), innate immune cells expanded soon after surgery, followed by a reduction of cells within the adaptive branch (FIG. 2).

In contrast, cell-signaling responses occurred early and simultaneously within both immune branches (FIG. 3). For instance, orchestrated changes in STAT3 and STAT5 signaling manifested within 1 h after surgery in CD14⁺MCs and CD4⁺ and CD8⁺ T cells. Our results challenge the view that innate immune responses to trauma precede adaptive responses. Our data dovetail with findings of a recent genomewide analysis of the leukocyte response to major trauma (Xiao et al. (2011) JEM 208, 2581-2590). In that study, up-regulation of genes associated with the innate immune branch and those encoding pro-inflammatory mediators, including STAT target genes, was concurrent with the suppression of genes associated with the adaptive immune branch including genes regulating T cell proliferation, antigen presentation, and apoptosis.

In CD14⁺ monocytes, STAT3 phosphorylation peaked 1 h after surgery in all patients and coincided with the de-phosphorylation of 10 signaling proteins, which formed four distinct modules (FIG. 3). The observed activation of STAT proteins in CD14⁺ monocytes within 24 h after surgery is consistent with reported trauma-induced increases in plasma cytokine IL-6 and IL-10, a key response to trauma. Unexpectedly, a biphasic response of several signaling pathways downstream of Toll-Like Receptors, which play a major role in innate immunity was observed (FIG. 3). The coordinated and sequential de-phosphorylation and phosphorylation of these proteins is reminiscent of oscillations in NF-κB nuclear translocation, which control the expression of NF-κB target genes. Oscillations in CREB and NF-κB signaling networks together with STAT3 signaling may drive the response of CD14⁺ monocytes to surgical trauma. Observed similarities in signaling activities among patients are indicative of a tightly regimented immune response to surgical trauma. However, the differences in the magnitude of such responses can account for differences in recovery from surgery.

Strikingly, inter-patient variability in phosphorylation of proteins within two signaling modules in CD14⁺ monocytes, those involving STAT3 (Module 4) and CREB and NF-κB (Module 1) (FIG. 3), strongly correlated with functional recovery and resolution of pain after surgery, suggesting that differential engagement of these signaling networks in CD14⁺ monocytes regulate important aspects of clinical recovery (FIG. 5). The most significant immune correlates of clinical recovery occurred in CD11b⁺CD14⁺HLADR^{low} monocytes (FIG. 5). A marked over-representation of this cell subset was observed at 24 h and 72 h after surgery (FIG. 2). Phenotypically CD11b⁺CD14⁺HLADR^{low} monocytes are remarkably similar to myeloid derived suppressor cells (MDSCs), which dramatically expand in a mouse model of surgery. In human malignancies CD11b⁺CD14⁺HLADR^{low} MDSCs proliferate and suppress T cell responses in a STAT3-dependent fashion. The observed preponderance of CD11b⁺CD14⁺HLADR^{low} cells after surgery and the strong correlation between STAT3 signaling in these cells and patients' global functional status strongly suggests that these cells regulate critical aspects of clinical recovery.

Previous studies had revealed a link between surgery-related inflammatory responses and clinical recovery; however, the immune features only explained a small fraction of variability in recovery rates and provided limited mechanistic insight. See, for example, Hall et al. (2001) British journal of anaesthesia 87, 537-542; and Rosenberg et al. supra. By contrast, single-cell mass cytometry revealed highly specific immune correlates accounting for 40-60% of variability in recovery rates (FIG. 5). Prior studies that relied on bulk analysis, precluded detailed sub-setting of cells, or could not measure functional attributes of rare cell subsets missed strong correlative signals. Notably, in the present work signaling responses in less than 2% of peripheral leukocytes determined a given clinical correlate.

The role of monocytes in clinical recovery from surgery and trauma is subject of significant interest. Application of mass cytometry at the bedside enabled identification of strong and specific immune correlates in CD14⁺ monocytes that accounted for 40-60% of patient-associated variability in recovery after PHA. Importantly, immune correlates pertained to the functional (i.e., signaling) state of CD14⁺ monocytes rather than their frequency. These data provide the first set of mechanistically based targets (including STAT3, CREB and NF-κB signaling) in immune cells to guide post-operative care in surgical patients. The diagnostic descriptors of the outcomes can be distilled into a total of six markers that are readily adaptable to a fluorescence-based flow cytometry test, to mass cytometry, and other such analyses. We expect the approach outlined here might eventually be used to distinguish aspects of the immune response that are misdirected or impaired after trauma and that might be targeted for the benefit of patients with predicted poor recovery.

TABLE 1
Demographic and clinical variables (n = 26)

| Demographics¹ | | |
|---|---|---|
| Gender (male/female) | 16/10 | |
| Race (Caucasian/ | 25/1 | |
| African American) | | |
| Age (year) | 59.5 (54.0-68.0) | |
| Body mass index (kg/m²) | 26.5 (24.4-28.1) | |
| Questionnaires² | Before surgery | 6 weeks after surgery |
| SRS | 62.3 (57.3-80.8) | 80.8 (67.1-86.8) |
| WOMAC | 131.5 (80.0-180.0) | 33.5 (11.0-51.0) |
| SF36 | | |
| PCS | 38.9 (21.9-42.1) | 41.5 (31.1-49.8) |
| MCS | 55.3 (39.7-59.6) | 60.3 (49.3-64.3) |
| BDI | 7.5 (3.0-11.0) | 5.5 (1.0-8.0) |
| POMS-A | | |
| Men | 5.5 (3.5-9.5) | 5.0 (4.0-6.8) |
| Women | 7.0 (5.0-14.0) | 4.0 (3.0-4.0) |
| Surgery | | |

| | |
|---|---|
| Duration (min) | 100 (85-119) |
| Blood loss (ml) | 250 (200-310) |
| Urine output (ml) | 200 (100-300) |
| Fluids | |
| Crystalloids (ml) | 1500 (1000-2000) |
| Colloids (ml) | 0 (0-0) |
| Blood products (ml) | 0 (0-0) |
| Time to discharge (days) | 3.1 (3.0-3.8) |
| Anesthesia³ | |
| Technique | |
| General (number of | 6 |
| patients) | |
| Spinal (number of patients) | 1 |
| General + Spinal (number | 19 |
| of patients) | |
| Volatile anesthetic | |
| Number of patients | 25 |
| MAC (%) | 0.5 (0.4-0.7) |
| Nitrous oxide | |
| Number of patients | 11 |
| MAC (%) | 0.5 (0.4-0.6) |
| Intrathecal medications | |
| Number of patients (%) | 20 |
| Bupivacaine (mg) | 11.3 (10.5-12.0) |
| Morphine (mg) | 0.2 (0.1-0.2) |
| Opioid use⁴ | |
| Intraoperative (mg) | 2.8 (1.5-3.8) |
| During hospital stay (mg) | 16.0 (13.1-27.4) |
| After discharge (mg) | 9.0 (5.5-16.9) |

¹Values indicate number of patients or median and interquartile range.
²SRS = Surgical Recovery Scale (0-100, minimal to maximal general function); WOMAC = Western Ontario and McMaster Universities Arthritis Index (0-240; minimal and maximal joint impairment); SF36 = Short Form Health Survey; PCS = Physical Component Score (normalized average and standard deviation in general population = 50 ± 10); MCS = Mental Component Score; BDI = Beck Depresiion Inventory (scores 0-13, 14-19, 20-28, and >28 = no, mild, moderate, and severe depression); POMS-A = Profile of Moods States Tension-Anxiety Scale (score >10 for men and >16 for women are clinically significant).
³MAC = Minimal Alveolar Concentration of average exposure during surgery.
⁴Milligram equivalent of intravenous hydromorphone; Dose during hospitalization is total cumulative dose; Dose after discharge is cumulative dose taken on survey days (13 days during observation from day 6 to 42).

TABLE 2
Antibody panels used for mass cytometry analysis.

| Mass-tagged antibody panel | | | | | Mass-tagged antibody panel | | | | |
|---|---|---|---|---|---|---|---|---|---|
| used in the preliminary analysis of 6 patients | | | | | used in the analysis of the subsequent 26 patients | | | | |
| | | | Phosphorylation | | | | | Phosphorylation | |
| Isotope | Antigen | Clone | site | Supplier | Isotope | Antigen | Clone | site | Supplier |
| In 113 | CD235ab | HIR2 | | Biolegend | In 113 | CD235ab | HIR2 | | Biolegend |
| In 113 | CD61 | VI-PL2 | | BD | In 113 | CD61 | VI-PL2 | | BD |
| La 139 | pSTAT3 | 4/P | pY705 | BD | In 115 | CD45 | HI30 | | |
| Pr 141 | CD7 | M-T701 | | BD | La 139 | pSTAT3 | 4/P | pY705 | BD |
| Nd 142 | CD19 | HIB19 | | DVS | Pr 141 | CD7 | M-T701 | | BD |
| Nd 143 | STAT1 | 4a | pY701 | BD | Nd 142 | CD19 | HIB19 | | DVS |
| Nd 144 | CD11b | ICRF44 | | DVS | Nd 143 | STAT1 | 4a | pY701 | BD |
| Nd 145 | CD4 | RPA-T4 | | DVS | Nd 144 | CD11b | ICRF44 | | DVS |
| Nd 146 | CD8a | RPA-T8 | | DVS | Nd 145 | CD4 | RPA-T4 | | DVS |
| Sm 147 | CD20 | 2H7 | | DVS | Nd 146 | CD8a | RPA-T8 | | DVS |
| Nd 148 | pCREB | 87G3 | pS133 | CST | Sm 147 | CD127 | HCD127 | | Biolegend |
| Sm 149 | STAT6 | 18 | pY641 | BD | Nd 148 | pCREB | 87G3 | pS133 | CST |
| Nd 150 | CCR7 | 150503 | | R&D | Sm 149 | pP65 | K10-895.12.50 | pS529 | BD |
| Eu 151 | CD123 | 6H6 | | DVS | Nd 150 | CCR7 | 150503 | | R&D |
| Sm 152 | PLCg2 | K86-689.37 | pY759 | BD | Eu 151 | CD123 | 6H6 | | DVS |
| Eu 153 | CD45RA | HI100 | | DVS | Sm 152 | PLCg2 | K86-689.37 | pY759 | BD |
| Sm 154 | CD45 | HI30 | | DVS | Eu 153 | CD45RA | HI100 | | DVS |
| Gd 158 | CD33 | WM53 | | Biolegend | Sm 154 | NkP44 | 253415 | | R&D |
| Tb 159 | CD11c | Bu15 | | DVS | Gd 156 | pP38 | 36/p38 | pT184/pY182 | BD |
| Gd 160 | CD14 | M5E2 | | DVS | Gd 158 | CD33 | WM53 | | Biolegend |
| Dy 164 | pSTAT5 | 47 | pY694 | BD | Tb 159 | CD11c | Bu15 | | DVS |
| Ho 165 | CD16 | 3G8 | | DVS | Gd 160 | CD14 | M5E2 | | DVS |
| Er 166 | STAT4 | 38 | pY693 | BD | Dy 162 | CD69 | FN50 | | DVS |
| Er 167 | CD27 | O323 | | DVS | Dy 164 | pSTAT5 | 47 | pY694 | BD |
| Er 168 | pERK | D13.14.4E | pT202/Y404 | CST | Ho 165 | CD16 | 3G8 | | DVS |
| Tm 169 | pP38 | 36/p38 | pT184/pY182 | BD | Er 166 | FoxP3 | PCH101 | | Ebioscience |
| Er 170 | CD3 | UCHT1 | | DVS | Er 167 | pMAPKAPK2 | 27B7 | pT334 | CST |
| Yb 171 | CD66 | CD66a-B1.1 | | DVS | Er 168 | pERK | D13.14.4E | pT202/Y404 | CST |
| Yb 172 | prpS6 | N7-548 | pS235/236 | BD | Tm 169 | CD25 | 2A3 | | DVS |
| Yb 174 | HLA-DR | L243 | | DVS | Er 170 | CD3 | UCHT1 | | DVS |
| Yb 176 | CD56 | HCD56 | | Biolegend | Yb 171 | CD66 | CD66a-B1.1 | | DVS |

| | | | | |
|---|---|---|---|---|
| Yb 172 | pS6 | N7-548 | pS235/236 | BD |
| Yb 174 | HLA-DR | L243 | | DVS |
| Yb 175 | CD56 | NCAM | | BD |
| Yb 176 | pP90RSK | D5D8 | pS380 | CST |

Listed are antibody-clones, metal isotopes, target-antigens, and distributors. Antibodies were chosen to identify major immune cell types in whole blood and to examine signaling pathways likely affected by surgery. All reagents were validated in whole blood samples. Panels of antibodies directed toward intracellular phospho-proteins differed moderately between the pilot and the main study as non-informative antibodies (pSTAT4, pSTAT6) were replaced with antibodies allowing more detailed examination of signaling pathways strongly affected by surgery (pMAPKAPK2, pP90RSK, pP65). Similarly, less informative cell surface antibodies (CD20, CD27) were replaced with antibodies to facilitate the gating of additional cell populations (CD25, CD127, NkP44, CD69, FoxP3).

TABLE 3
SAM analysis of intracellular signaling responses over time.

| | | |
|---|---|---|
| signaling | Score(d) | q-value(%) |

Upregulated signaling responses (1 h)

| | | |
|---|---|---|
| CD14+MCs_pSTAT3 | 10.24 | 0.00 |
| CD4Tcells_pSTAT3 | 6.59 | 0.00 |
| cDCs_pSTAT3 | 6.01 | 0.00 |
| neutrophils_pSTAT3 | 5.51 | 0.00 |
| CD4Tcells_pSTAT5 | 4.75 | 0.00 |
| CD4Tcells_pERK | 4.41 | 0.00 |
| CD8Tcells_pERK | 4.15 | 0.00 |
| CD8Tcells_pSTAT3 | 3.98 | 0.00 |
| neutrophils_pPLCg2 | 3.93 | 0.00 |
| pDCs_pS8 | 3.15 | 0.00 |
| pDCs_pSTAT3 | 3.08 | 0.00 |
| neutrophils_pSTAT5 | 2.68 | 0.00 |

Downregulated signaling responses (1 h)

| | | |
|---|---|---|
| CD14+MCs_pMK2 | −7.47 | 0.00 |
| Bcells_pMK2 | −7.37 | 0.00 |
| cDCs_pMK2 | −7.19 | 0.00 |
| NKcells_pMK2 | −6.95 | 0.00 |
| CD14+MCs_pCREB | −6.47 | 0.00 |
| CD14+MCs_pERK | −5.78 | 0.00 |
| CD14+MCs_pP90RSK | −5.53 | 0.00 |
| CD8Tcells_pMK2 | −5.12 | 0.00 |
| CD4Tcells_pMK2 | −5.01 | 0.00 |
| CD14+MCs_pP38 | −4.75 | 0.00 |
| CD14+MCs_pS8 | −4.54 | 0.00 |
| CD14+MCs_pPLCg2 | −4.49 | 0.00 |
| NKcells_pP90RSK | −4.35 | 0.00 |
| cDCs_pERK | −4.04 | 0.00 |
| CD14+MCs_pSTAT5 | −3.81 | 0.00 |
| cDCs_pP90RSK | −3.81 | 0.00 |
| cDCs_pCREB | −3.65 | 0.00 |
| neutrophils_pERK | −3.63 | 0.00 |
| neutrophils_pCREB | −3.22 | 0.00 |
| CD14+MCs_pNFkB | −3.20 | 0.00 |
| NKcells_pCREB | −3.18 | 0.00 |
| pDCs_pPLCg2 | −2.81 | 0.00 |
| cDCs_pPLCg2 | −2.77 | 0.00 |
| NKcells_pPLCg2 | −2.75 | 0.00 |
| pDCs_pMK2 | −2.48 | 0.00 |
| NKcells_pSTAT5 | −2.25 | 0.00 |
| Bcells_pS8 | −2.19 | 0.00 |
| CD8Tcells_pCREB | −2.17 | 0.00 |
| pDCs_pSTAT5 | −2.12 | 0.00 |
| CD4Tcells_pCREB | −2.09 | 0.00 |
| cDCs_pS8 | −1.87 | 0.69 |
| CD14+MCs_pSTAT1 | −1.85 | 0.69 |
| neutrophils_pSTAT1 | −1.82 | 0.69 |
| Bcells_pPLCg2 | −1.80 | 0.69 |
| Bcells_pCREB | −1.72 | 0.69 |

Upregulated signaling responses (24 h)

| | | |
|---|---|---|
| CD4Tcells_pSTAT3 | 13.80 | 0.00 |
| cDCs_pSTAT3 | 9.94 | 0.00 |
| CD4Tcells_pSTAT5 | 9.14 | 0.00 |
| CD14+MCs_pSTAT1 | 9.13 | 0.00 |
| CD14+MCs_pSTAT3 | 7.54 | 0.00 |
| pDCs_pSTAT3 | 6.77 | 0.00 |
| neutrophils_pSTAT3 | 6.71 | 0.00 |
| CD8Tcells_pSTAT3 | 6.34 | 0.00 |
| cDCs_pSTAT1 | 5.82 | 0.00 |
| CD8Tcells_pSTAT5 | 5.72 | 0.00 |
| cDCs_pNFkB | 5.62 | 0.00 |
| CD4Tcells_pNFkB | 5.57 | 0.00 |
| neutrophils_pSTAT5 | 5.15 | 0.00 |
| CD4Tcells_pSTAT1 | 5.07 | 0.00 |
| cDCs_pS8 | 4.55 | 0.00 |
| CD8Tcells_pNFkB | 3.90 | 0.00 |
| CD14+MCs_pNFkB | 3.76 | 0.00 |
| pDCs_pNFkB | 3.32 | 0.00 |
| CD14+MCs_pERK | 3.19 | 0.00 |
| neutrophils_pPLCg2 | 3.15 | 0.00 |
| cDCs_pSTAT5 | 3.04 | 0.00 |
| CD4Tcells_pERK | 3.04 | 0.00 |
| CD8Tcells_pSTAT1 | 3.03 | 0.00 |
| cDCs_pERK | 2.79 | 0.00 |
| CD8Tcells_pERK | 2.57 | 0.00 |
| Bcells_pSTAT1 | 2.39 | 0.00 |

Downregulated signaling responses (24 h)

| | | |
|---|---|---|
| Bcells_pMK2 | −6.03 | 0.00 |
| NKcells_pCREB | −5.42 | 0.00 |
| NKcells_pMK2 | −4.67 | 0.00 |
| CD14+MCs_pCREB | −4.03 | 0.00 |
| CD4Tcells_pMK2 | −3.98 | 0.00 |
| CD8Tcells_pCREB | −3.78 | 0.00 |
| Bcells_pP90RSK | −3.65 | 0.00 |
| CD8Tcells_pMK2 | −3.60 | 0.00 |
| pDCs_pMK2 | −3.38 | 0.00 |
| cDCs_pMK2 | −2.98 | 0.00 |

Upregulated signaling responses (72 h)

| | | |
|---|---|---|
| CD4Tcells_pSTAT3 | 14.93 | 0.00 |
| CD14+MCs_pSTAT1 | 12.19 | 0.00 |
| NKcells_pP90RSK | 7.95 | 0.00 |
| CD4Tcells_pSTAT5 | 7.89 | 0.00 |
| CD8Tcells_pSTAT3 | 6.73 | 0.00 |
| cDCs_pSTAT1 | 6.10 | 0.00 |
| CD14+MCs_pNFkB | 5.77 | 0.00 |
| cDCs_pNFkB | 5.43 | 0.00 |
| cDCs_pSTAT3 | 5.28 | 0.00 |
| neutrophils_pSTAT1 | 4.71 | 0.00 |
| CD4Tcells_pERK | 4.70 | 0.00 |
| CD8Tcells_pSTAT5 | 4.64 | 0.00 |
| CD4Tcells_pNFkB | 4.53 | 0.00 |
| CD14+MCs_pERK | 4.36 | 0.00 |
| pDCs_pSTAT3 | 4.23 | 0.00 |
| CD8Tcells_pERK | 4.15 | 0.00 |
| CD4Tcells_pSTAT1 | 4.14 | 0.00 |
| neutrophils_pSTAT5 | 4.11 | 0.00 |
| CD14+MCs_pS8 | 3.96 | 0.00 |
| cDCs_pS8 | 3.78 | 0.00 |
| cDCs_pERK | 3.51 | 0.00 |
| CD8Tcells_pSTAT1 | 3.49 | 0.00 |
| neutrophils_pNFkB | 3.39 | 0.00 |
| CD14+MCs_pSTAT3 | 3.36 | 0.00 |
| neutrophils_pERK | 3.32 | 0.00 |
| CD14+MCs_pP90RSK | 3.18 | 0.00 |
| neutrophils_pSTAT3 | 3.10 | 0.00 |
| neutrophils_pP90RSK | 2.89 | 0.00 |
| neutrophils_pPLCg2 | 2.77 | 0.00 |
| CD8Tcells_pNFkB | 2.36 | 0.00 |
| NKcells_pSTAT1 | 2.17 | 0.00 |
| neutrophils_pP38 | 2.11 | 0.00 |
| cDCs_pP90RSK | 2.03 | 0.68 |
| neutrophils_pS8 | 1.94 | 0.68 |
| cDCs_pSTAT5 | 1.91 | 0.68 |
| NKcells_pSTAT5 | 1.90 | 0.68 |
| Bcells_pSTAT1 | 1.90 | 0.68 |
| CD4Tcells_pP38 | 1.87 | 0.68 |
| pDCs_pSTAT1 | 1.84 | 0.68 |
| cDCs_pP38 | 1.77 | 0.68 |
| CD14+MCs_pP38 | 1.74 | 0.68 |
| CD14+MCs_pMK2 | 1.74 | 0.68 |
| NKcells_pNFkB | 1.65 | 0.68 |

Downregulated signaling responses (72 h)

| | | |
|---|---|---|
| CD14+MCs_pPLCg2 | −4.32 | 0.00 |
| Bcells_pP90RSk | −3.62 | 0.00 |
| Bcells_pMK2 | −3.12 | 0.00 |
| cDCs_pPLCg2 | −3.10 | 0.00 |
| Nkcells_pMK2 | −3.04 | 0.00 |

Upregulated signaling responses (6 wks)

| | | |
|---|---|---|
| cDCs_pSTAT1 | 2.55 | 0.00 |
| pDCs_pSTAT1 | 2.04 | 0.00 |
| pDCs_pS8 | 1.97 | 0.00 |
| CD4Tcells_pSTAT1 | 1.86 | 0.00 |

Significant changes of intracellular signaling responses 1 h, 24 h, 72 h and 6 wks after surgery were determined with SAM Two class paired. Signaling responses are defined as the change from baseline of the median signal intensity associated with a phospho-protein in an immune cell-subset. The tables separately list up-regulated and down-regulated signaling responses detected at a false discovery rate of q < 0.01. Results are ranked according to their statistical significance (d-score). Positive and negative d-scores indicate the direction of change.

TABLE 4
Immune features correlating with clinical parameters of surgical recovery.

| Clinical parameter | Immune feature type | Immune feature | Cell subset | R |
|---|---|---|---|---|
| Time to 50% global function | Signaling property | 1 h vs 24 h cluster 519927 (A1), pSTAT3 | CD14⁺ HLA-DR^{med} | 0.80 |
| Time to 50% global function | Signaling property | 1 h vs 24 h cluster 519972 (A), pSTAT3 | CD14⁺ HLA-DR^{low} | 0.74 |
| Time to 50% global function | Signaling property | 1 h vs 24 h cluster 519978 (A2), pSTAT3 | CD14⁺ HLA-DR^{low} | 0.73 |
| Time to 50% global function | Signaling property | 1 h vs 24 h cluster 519805 (A3), pSTAT3 | CD14⁺ HLA-DR^{med} | 0.72 |
| Time to mild pain | Signaling property | BL vs 1 h cluster 519930 (C), pNFkB | CD14⁺ HLA-DR^{hi} | 0.71 |
| Time to mild FI of the hip | Signaling property | BL vs 1 h cluster 519883 (B), pCREB | CD14⁺ HLA-DR^{low} | 0.66 |

Significant immune features were cell abundance (percentage of total CD45+CD66− cells) and signaling responses of eleven intracellular phospho-proteins within a cell cluster. Six significant correlations were detected between immune features and parameters of clinical recovery at a false discovery rate of q < 0.01 (SAM Quantitative). Results are ranked by descending Spearman's correlation coefficients (R). All significant correlations were signaling responses in clusters within the CD14+MC compartment. BL = base line, FI = functional impairment of the hip.

TABLE 5
Immune feature correlations with clinical parameter of surgical recovery corrected for clinical covariates.

| Clinical parameter | Immune feature | Age | Sex | BMI | Spinal | Duration | SBL |
|---|---|---|---|---|---|---|---|
| Time to 50% global function | 1 h vs 24 h cluster A1, pSTAT3 | 0.79 *** | 0.72 *** | 0.75 *** | 0.79 *** | 0.76 *** | 0.78 *** |
| Time to 50% global function | 1 h vs 24 h cluster A, pSTAT3 | 0.82 *** | 0.71 *** | 0.73 *** | 0.74 *** | 0.73 *** | 0.77 *** |
| Time to 50% global function | 1 h vs 24 h cluster A3, pSTAT3 | 0.75 *** | 0.70 *** | 0.72 *** | 0.75 *** | 0.75 *** | 0.75 *** |
| Time to 50% global function | 1 h vs 24 h cluster A2, pSTAT3 | 0.78 *** | 0.66 ** | 0.70 *** | 0.70 *** | 0.67 ** | 0.73 *** |
| Time to mild pain | BL vs 1 h cluster B, pCREB | 0.64 ** | 0.66 ** | 0.69 *** | 0.64 ** | 0.67 ** | 0.62 ** |
| Time to mild FI of the hip | BL vs 1 h cluster C, pNFkB | 0.71 *** | 0.71 *** | 0.71 *** | 0.69 *** | 0.70 *** | 0.61 ** |

| Clinical parameter | Immune feature | SRS, BL | Pain, BL | Hip function, BL |
|---|---|---|---|---|
| Time to 50% global function | 1 h vs 24 h cluster A1, pSTAT3 | 0.72 *** | 0.78 *** | 0.78 *** |
| Time to 50% global function | 1 h vs 24 h cluster A, pSTAT3 | 0.68 *** | 0.75 *** | 0.74 *** |
| Time to 50% global function | 1 h vs 24 h cluster A3, pSTAT3 | 0.69 *** | 0.76 *** | 0.74 *** |
| Time to 50% global function | 1 h vs 24 h cluster A2, pSTAT3 | 0.66 ** | 0.71 *** | 0.70 *** |
| Time to mild pain | BL vs 1 h cluster B, pCREB | 0.60 ** | 0.66 ** | 0.65 ** |
| Time to mild FI of the hip | BL vs 1 h cluster C, pNFkB | 0.70 *** | 0.70 *** | 0.70 *** |

The influence of clinical covariates on significant correlations between clinical recovery parameters and immune features were assessed with partial correlation analysis. Clinical covariates included age, sex, body mass index, (BMI), use of a neuraxial anesthetic technique (spinal), duration of surgery (duration), surgical blood loss (SBL), and preoperative scores on the Surgical Recovery Scale (SRS, BL), WOMAC pain scale (pain, BL), and WOMAC function scale (hip function, BL). Shown are the Spearman ranked correlation coefficient between residuals (R) accounting for the clinical covariate and associated p-values (p). All correlations remained significant when controlling for clinical covariates.
*** p ≦ 0.0001,
** p p ≦ 0.001.

Subjects.

The study was approved by the Institutional review Board of Stanford University School of Medicine and registered with ClinicalTrials.gov (NCT01578798). Patients scheduled for primary hip arthroplasty were recruited from the Arthritis and Joint Replacement Clinic in the Department of Orthopedic Surgery at Stanford University School of Medicine. A total of 251 patients were screened, 81 were approached for consent, 50 were consented, 39 were actively enrolled, and 32 completed the study (FIG. 9). Inclusion criteria were: 1) scheduled for primary hip arthroplasty, 2) age 18-90 years, 3) fluent in English, and 4) willing and able to sign informed consent and the Health Insurance Portability and Accountability Act (HIPAA) authorization. Exclusion criteria were: 1) any systemic disease or medication that might compromise the immune system, 2) diagnosis of cancer within the last 5 years, 3) psychiatric, immunological, and neurological conditions that would interfere with the collection and interpretation of study data, 4) pregnancy, and 5) any other conditions that, in the opinion of the investigators, may have compromised a participant's safety or the integrity of the study.

Study Protocol.

Assessments were made 1 h before and 1, 24, 48, and 72 h after surgery and every third day from day 6 to day 42. Clinical outcomes were captured with the Surgical Recovery Scale (SRS; 0-100=worst/best function), an adapted Western Ontario and McMaster Universities Arthritis Index (WOMAC) pain scale (WOMAC-P; 0-40=no/worst imaginable pain), and an adapted WOMAC function scale (WOMAC-F; 0-60=no/severe functional impairment). Adapted versions were used because not all questions applied to the postoperative setting. Daily opioid consumption was captured as intravenous hydromorphone equivalents. Full versions of the WOMAC, the Short Form Health Survey (SF-36), the Profile of Moods States Tension-Anxiety Scale (POMS-A) and the Beck Depression Inventory (BDI) were completed at the beginning and end of the study.

Clinical Data.

Results are presented as medians and interquartile ranges. Recovery of global functioning (SRS) was quantified as the time required to half maximum recovery (SRS-t_{1/2}), defined by a surgical recovery index (SRI) equivalent to the sum of the minimum SRI after surgery and half of the difference between the preoperative SRI and the minimum SRI. SRS-t_{1/2} was more sensitive than time to full recovery as the latter parameter was affected by ceiling effects. Recovery from pain was quantified as the time required to achieve a WOMAC-P≦12 (T₁₂). WOMACP was composed of four scores (0-10=no/most imaginable pain) to quantify pain at night, at rest, when bearing weight, and during walking. A cumulative score of 12 indicates transition from mild to moderate pain across the conditions. Recovery of hip function was quantified as the time required to achieve a WOMAC-F≦18 (T₁₈). WOMAC-F was composed of six scores (0-10=no/severely impaired) to quantify function in the affected hip when lying in bed, rising from bed, sitting, rising from sitting, standing, and walking on flat surface. A cumulative score of 18 indicates transition from moderate to mild functional impairment across the conditions.

Whole Blood Processing.

Blood samples were resuspended in stabilizing buffer (Smarttube, Inc.) within 30 min of phlebotomy and stored at −80° C. Samples were thawed on the day of processing. Red blood cells were lysed using a hypotonic buffer. Peripheral blood leukocytes were washed and resuspended in cell staining media.

In Vitro Stimulation of Whole Blood Samples.

Stimulations were performed for antibody validation (FIG. 6b). Samples were incubated with PBS, interleukin cocktail (100 ng/mL IL-2 [BD Biosciences]; 100 ng/mL IL-6 [BD Pharmingen]; 20 ng/mL IFN<[Sigma-Aldrich]; 2 ng/mL GMCSF [PeproTech]), 80 nM phorbol 12-myristate 13-acetate/1.3 μM ionomycin, [Ebioscience]), or 0.5 mM activated sodium orthovanadate [Calbiochem]) for 15 min at 37° C. Blood samples were resuspended, cooled to 4° C. and stored at −80° C.

Antibodies.

Antibodies were chosen to facilitate the identification of major immune cell types in whole blood (FIG. 7) as well as to measure immune signaling pathways likely to be affected by surgery. The antibodies used for the six-patient pilot study are listed in Table 2, a subset of these were used for experiments described in FIG. 7. An updated panel (Table 2) was used for the 26-patient study. This panel substituted signaling antibodies that showed little change in the six patients (pSTAT4, pSTAT6) with antibodies that reflected pathways that changed more substantially (pMAPKAPK2, pP90RSK, pNF-κB (p65-RelA)). Phenotypic antibodies were updated by substituting non-functional or redundant markers (CD20, CD27) with markers that facilitate the gating of additional cell populations (CD25, CD127, NkP44, CD69, FoxP3). A subset of the antibodies was obtained pre-labeled by DVS Sciences (DVS Sciences); others were metal-labeled as described by Bendall et al. (2011) Science 332, 687-696. Briefly, antibodies were obtained in carrier protein-free PBS and labeled using the MaxPAR antibody conjugation kit (DVS Sciences) according to the manufacturer's protocol. All metal-labeled antibodies were diluted based on their percent yield by measurement of absorbance at 280 nm to 0.2 mg/mL in Candor PBS Antibody Stabilization solution (Candor Biosciences) for storage at 4° C.

Barcoding.

Reagents were prepared according to the procedure described in Bodenmiller et al. (2012) Nature biotechnology. Two molar equivalents of maleimido-mono-amide-DOTA (Macrocyclics, Inc.) were added to palladium 102, 104, 105, 106, 108, 110, each contained in 20 mM ammonium acetate at pH 6.0. Solutions were immediately lyophilized, and solids were dissolved in dimethyl sulfoxide (DMSO) to 10 mM for storage at −20° C. Each well of a barcoding plate contained a unique combination of three palladium isotopes at 200 nM in DMSO.

Cell Barcoding and Antibody Staining.

Time points from the same patient (BL, 1 h, 24 h, 72 h, 6 weeks) were barcoded and processed simultaneously. To protect against potential batch effects, all findings are quantified as relative changes between time points when comparing patients. Cells were barcoded with alterations for pre-permeabilization. Cells were transferred into a deepwell block and washed once with Cell Staining Media (CSM, PBS with 0.5% BSA, 0.02% NaN₃), once with PBS, then once with 0.02% saponin in PBS leaving cells in 100 μL residual volume. The barcoding plate was thawed on ice, and 1 mL 0.02% saponin/PBS was added to each well. Aliquots were transferred to cells, and samples were incubated at room temperature for 15 min, washed twice with CSM, and pooled into one FACS tube for staining with metal-labeled antibodies. Cells were washed once with CSM and then incubated for 10 min at room temperature with one test of FcX block (Biolegend) to block non-specific Fc binding. Cells were stained with the surface antibody cocktail for 30 min and washed once with CSM. Cells were permeabilized with 1 mL of methanol for 10 min on ice. Cells were washed twice with PBS and once with CSM and incubated with the intracellular antibody cocktail for 30 min at room temperature. Cells were washed once with CSM then incubated overnight at 4° C. with an iridium-containing intercalator from DVS Sciences in PBS with 1.6% formaldehyde. Cells were washed twice with CSM, once with water, and then resuspended in a solution of normalization beads as described by Finck et al. (2013) Cytometry. Part A: the journal of the International Society for Analytical Cytology 83, 483-494. Cells were filtered through a 35-μm membrane prior to mass cytometry analysis.

Mass Cytometry.

Stained cells were analyzed on a mass cytometer (CyTOF, DVS Sciences) at an event rate of 400-500 cells per second. Data files for each barcoded sample were concatenated using an in-house script. The data were normalized using Normalizer v0.1 MCR. Files were de-barcoded using the Matlab Debarcoder Tool. For gating see FIG. 7.

Statistical Analyses of Molecular Parameters

An inverse hyperbolic sine transformation was applied to analyze protein phosphorylation data. The difference of the median of the transformed values between baseline and 1 h, 24 h, 72 h, and 6 weeks after surgery is reported as the arcsinh ratio. Significant changes in cell frequency and phosphorylation state were inferred with SAM₃₅, using the “samr” package in R. SAM Two class paired was performed for hand-gated data. Significance was inferred for a false discovery rate <1% (FDR, q<0.01).

Correlation Network Analysis.

Monocyte signaling responses from all time points were used to generate a Pearson correlation matrix, which was clustered using single-linkage clustering (FIG. 13). Clusters were collapsed into a module when the within-cluster correlation exceeded 0.7 (FIG. 3d). Correlations between two modules were calculated as the average of the correlation between the points in the two modules (FIG. 3e).

Clustering.

Hierarchical clustering using Ward's linkage and Euclidean distance was performed on CD45⁺CD66⁻ cells using R (FIG. 2, 5). Cells were clustered based on the expression of CD7, CD19, CD11b, CD4, CD8, CD127, CCR7, CD123, CD45RA, CD33, CD11c, CD14, CD16, FoxP3, CD25, CD3, HLA-DR, and CD56. Ten thousand events were sampled from each patient sample for clustering. Clusters containing at least 1% of all clustered cells are graphically displayed. Data from timepoints that were included in the same SAM analysis were clustered together to enable comparison of clusters between timepoints.

Correlation Analyses of Molecular and Clinical Parameters.

Cell subsets were identified using hierarchical clustering as described in the “clustering” section. For each cluster in each patient, cluster abundances and the median value of 11 phospho-proteins were calculated. Associations between clinical endpoints and cluster properties were identified using the SAM Quantitative method. Repeated runs of the analysis with identical parameters confirmed that results were reproducible. Partial correlation was performed by correlating the residuals from (1) the correlation of the clinical covariate with the immune feature and (2) the correlation between the clinical covariate and the clinical index. Analysis was performed in the R software environment. P-values from this analysis were compared to the p-values for the immune feature correlation with the clinical index and are listed in Table 5.

Visualizations.

Visualizations of the cluster hierarchy plots and histograms were created in the R software environment. Correlation networks were visualized using TreeView software. Heatmaps were created using the ggplot2 package in R. Additional graphs were created using Prism (Graphpad).

### Example 2

### Ex Vivo Testing

The ability to elicit responses as described in Example 1 were tested in an ex vivo system. Such responses allow detection of patient differences in immune responses to surgery that are associated with recovery.

A series of stimulations to peripheral blood samples taken from surgery patients at pre-operative baseline were performed, including contacting the blood sample with one or more of cytokines, growth factors, and bacterial antigens, in an effort to elicit a cellular inflammatory response ex vivo. The baseline sample from each patient was divided into five aliquots and contacted with either IL6, IL10, IL2+GMCSF, or LPS, leaving one sample untreated. Samples were incubated at 37° C. for 15 minutes, following a fixation for 10 minutes, and then frozen in the fixation/stabilization buffer. Samples were then processed for mass cytometry as described in Example 1 and FIG. 1.

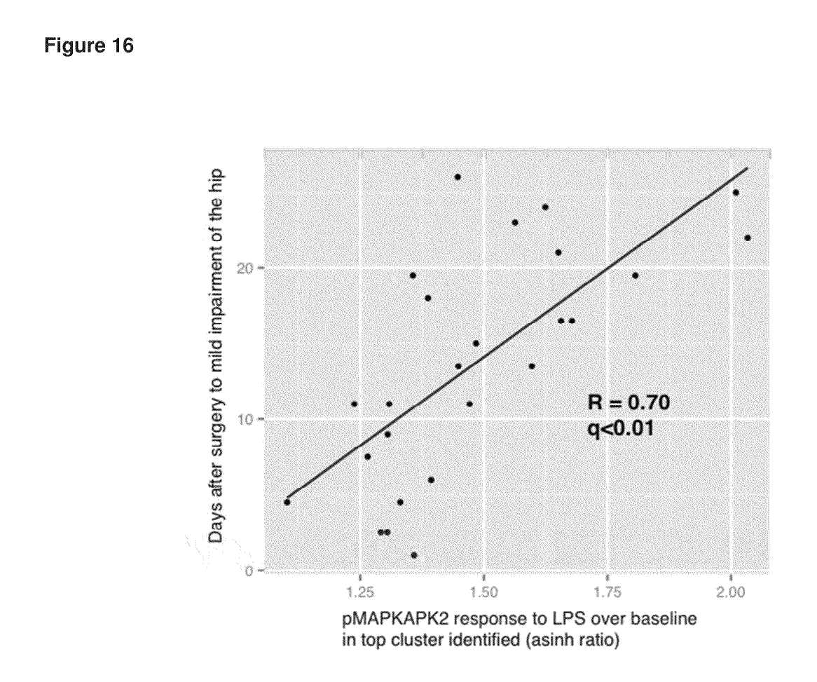

Using the computational method described in Example 1, (hierarchical clustering, feature extraction, and SAM), we detected 13 clusters whose pMAPKAPK2 activation in response to LPS stimulation relative to the signal from the untreated sample strongly correlated with time to mild impairment of the hip (R=0.63-0.70, q<0.01, FIG. 16). These clusters all had a monocyte phenotype (CD33⁺CD11b⁺CD14⁺HLADR⁺), and were validated by manual gating (R=0.69).

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A method for assessing prognosis for time to recovery for an individual following surgery, comprising:
obtaining a cellular biological sample for analysis comprising immune cells from a patient contemplating or undergoing surgery,
measuring single cell levels of signaling proteins in immune cell subset(s);
determining whether changes in levels of activated signaling proteins associated with time to recovery are present; and
providing an assessment of the patient's prognosis for time to recovery.

2. The method of claim 1, wherein the cellular biological sample is a blood sample.

3. The method of claim 2, wherein the sample is obtained prior to surgery; and is contacted ex vivo with an stimulating agent in an effective dose and for a period of time sufficient to activate monocytes in the sample.

4. The method of claim 3, wherein the stimulating agent is a TLR4 agonist.

5. The method of claim 4, wherein the TLR4 agonist is LPS.

6. The method of claim 3, wherein the period of time is from about 5 minutes to about 24 hours.

7. The method of claim 1, wherein the sample for analysis is obtained within about 72 hours following surgery.

8. The method of claim 1, wherein the sample for analysis is obtained within about 24 hours following surgery.

9. The method of claim 1, wherein the sample for analysis is obtained within about 3 hours following surgery.

10. The method of claim 1, wherein the levels of activated signaling proteins associated with time to recovery are compared to reference levels.

11. The method of claim 10, wherein the reference level is obtained from pre-surgery control sample from the individual.

12. The method of claim 10, wherein the reference level is obtained from a sample immediately post-surgery from the individual.

13. The method of claim 1, wherein the activated signaling protein is one or more of pSTAT3, pSTAT1, pCREB, pSTAT6, pPLCγ2, pSTAT5, pSTAT4, pSTAT6, pERK, pP38, prpS6, pNF-κB (p65), pMAPKAPK2, pP90RSK, and a signaling molecule within the TLR4 pathway.

14. The method of claim 13, wherein the activated signaling protein is one or more of pSTAT3, pSTAT1, pCREB, pERK, and pNF-κB (p65).

15. The method of claim 14, wherein the activated signaling protein is one or more of pSTAT3, pCREB, and pNF-κB (p65).

16. The method of claim 15, wherein activated signaling proteins are each of pSTAT3, pCREB, and pNF-κB (p65).

17. The method of claim 1, wherein immune cells in the biological sample for analysis are phenotyped by cell surface markers.

18. The method of claim 17, wherein the cell surface markers are one or more of CD3, CD7, CD14, CD66, HLA-DR, CD11b, CD11c, CD33, CD45, CD235, CD61, CD19, CD4, CD8, CD123, and CCR7.

19. The method of claim 17, wherein the cell surface markers are one or more of CD3, CD14, CD66, HLA-DR, and CD11b.

20. The method of claim 17, wherein at least one cell surface marker is CD14.

21. The method of claim 20, wherein analysis is gated on CD14+ monocytes.

22. The method of claim 21, wherein analysis is gated on CD14+ monocytes subsets with high HLA-DR expression.

23. The method of claim 21, wherein analysis is gated on CD14+ monocytes subsets with low HLA-DR expression.

24. The method of claim 18, wherein analysis is gated on CD4+ T cells.

25. The method of claim 18, wherein analysis is gated on CD8+ T cells.

26. The method of claim 1, wherein an increase in pSTAT3 levels in CD14⁺ monocytes after about 4 to 48 hours following surgery, compared to a reference level immediately following surgery, is indicative that an individual will require a longer period of time to achieve recovery, as assessed by time to 50% global functioning.

27. The method of claim 1, wherein an increase in pCREB levels in CD14⁺ monocytes immediately following surgery compared to a pre-surgery reference level, is indicative that an individual will require a longer period of time to achieve recovery, as assessed, by time to mild functional impairment.

28. The method of claim 1, wherein an increase in pNF-κB levels in CD14⁺ monocytes immediately following surgery compared to a pre-surgery reference level, is indicative that an individual will require a longer period of time to achieve recovery, as assessed, by time to mild pain.

29. The method of claim 1, wherein treatment of the individual post-surgery is made in accordance with the prognosis.

30. The method of claim 1, wherein measuring single cell levels of activated signaling proteins in immune cell subset(s) is performed by contacting the sample with labeled affinity reagents specific for the activated signaling protein.

31. The method of claim 30, wherein analysis is performed by flow cytometry.

32. The method of claim 31, wherein the label is fluorescent.

33. The method of claim 31, wherein the label is an isotope label.

34. A kit for use in the method of claim 1.

35. The kit of claim 34, comprising affinity reagents that specifically identify one or more cells and signaling proteins indicative of the time to recovery status of the patient.

36. The kit of claim 34, wherein the affinity reagents comprise one or more of reagents that specifically bind to pNF-κB (pP65), pCREB, and/or pSTAT3.

37. The kit of claim 34, wherein the affinity reagents comprise a reagent that specifically binds to CD14.

38. The kit of claim 34, further comprising affinity reagents specific for one or more of CD66, CD3, CD11b, and HLA-DR.

39. The kit of claim 34, further comprising an stimulating agent.

40. The kit of claim 39, wherein the stimulating agent is a TLR4 agonist.

41. The kit of claim 34, further comprising a system for analysis.

42. The kit of claim 41, wherein the system for analysis includes a software component.
